(19) United States
(12) Patent Application Publication
(10) Pub. No.: US 2008/0242949 A1
(43) Pub. Date: Oct. 2, 2008

(54) Computational user-health testing

(75) Inventors: Edward K.Y. Jung, Bellevue, WA (US); Eric C. Leuthardt, St. Louis, MO (US); Royce A. Levien, Lexington, MA (US); Robert W. Lord, Seattle, WA (US); Mark A. Malamud, Seattle, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/804,304

(22) Filed: May 15, 2007

Related U.S. Application Data
(63) Continuation-in-part of application No. 11/731,745, filed on Mar. 30, 2007.
Application No. 11/731,745 is a continuation-in-part of application No. 11/731,778, filed on Mar. 30, 2007.
Application No. 11/731,778 is a continuation-in-part of application No. 11/731,801, filed on Mar. 30, 2007.

(51) Int. Cl. A61B 5/00; G01N 33/48

(52) U.S. Cl. 600/300; 702/19

(57) ABSTRACT

Methods, apparatuses, computer program products, devices and systems are described that carry out implementing in at least one device at least one user-health test function that is structurally distinct from at least one application whose primary function is different from symptom detection, the at least one application being operable on the at least one device; obtaining user data in response to an interaction between a user and the at least one application; and presenting an output of the at least one user-health test function at least partly based on the user data.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 11/731,745, entitled EFFECTIVE RESPONSE PROTOCOLS FOR HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,778, entitled CONFIGURING SOFTWARE FOR EFFECTIVE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.
For purposes of the USPTO extra-statutory requirements, the present application constitutes a continuation-in-part of U.S. patent application Ser. No. 11/731,801, entitled EFFECTIVE LOW PROFILE HEALTH MONITORING OR THE LIKE, naming Edward K. Y. Jung; Eric C. Leuthardt; Royce A. Levien; Robert W. Lord; and Mark A. Malamud as inventors, filed 30 Mar. 2007 which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

This description relates to data capture and data handling techniques.

### SUMMARY

An embodiment provides a method. In one implementation, the method includes but is not limited to implementing in at least one device at least one user-health test function that is structurally distinct from at least one application whose primary function is different from symptom detection, the at least one application being operable on the at least one device; obtaining user data in response to an interaction between a user and the at least one application; and presenting an output of the at least one user-health test function at least partly based on the user data. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

An embodiment provides a system. In one implementation, the system includes but is not limited to means for implementing in at least one device at least one user-health test function that is structurally distinct from at least one application whose primary function is different from symptom detection, the at least one application being operable on the at least one device; means for obtaining user data in response to an interaction between a user and the at least one application; and means for presenting an output of the at least one user-health test function at least partly based on the user data. In addition to the foregoing, other system aspects are described in the claims, drawings, and text forming a part of the present disclosure.

An embodiment provides a computer program product. In one implementation, the computer program product includes but is not limited to a signal-bearing medium bearing (a) one or more instructions for implementing in at least one device at least one user-health test function that is structurally distinct from at least one application whose primary function is different from symptom detection, the at least one application being operable on the at least one device; (b) one or more instructions for obtaining user data in response to an interaction between a user and the at least one application; and (c) one or more instructions for presenting an output of the at least one user-health test function at least partly based on the user data. In addition to the foregoing, other computer program product aspects are described in the claims, drawings, and text forming a part of the present disclosure.

An embodiment provides a system. In one implementation, the system includes but is not limited to a computing device and instructions. The instructions when executed on the computing device cause the computing device to (a) implement in at least one device at least one user-health test function that is structurally distinct from at least one application whose primary function is different from symptom detection, the at least one application being operable on the at least one device; (b) obtain user data in response to an interaction between the user and the at least one application; and (c) present an output of the at least one user-health test function at least partly based on the user data. In addition to the foregoing, other system aspects are described in the claims, drawings, and text forming a part of the present disclosure.

In one or more various aspects, related systems include but are not limited to computing means and/or programming for effecting the herein-referenced method aspects; the computing means and/or programming may be virtually any combination of hardware, software, and/or firmware configured to effect the herein-referenced method aspects depending upon the design choices of the system designer.

In addition to the foregoing, various other method and/or system and/or program product aspects are set forth and described in the teachings such as text (e.g., claims and/or detailed description) and/or drawings of the present disclosure.

The foregoing is a summary and thus contains, by necessity, simplifications, generalizations and omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE DRAWINGS

With reference now to FIG. 1, shown is an example of a user interaction and data processing system in which embodiments may be implemented, perhaps in a device, which may serve as a context for introducing one or more processes and/or devices described herein.

FIG. 2 illustrates certain alternative embodiments of the data capture and processing system of FIG. 1.

With reference now to FIG. 3, shown is an example of an operational flow representing example operations related to computational user-health testing, which may serve as a context for introducing one or more processes and/or devices described herein.

FIG. 4 illustrates an alternative embodiment of the example operational flow of FIG. 3.

FIG. 5 illustrates an alternative embodiment of the example operational flow of FIG. 3.

FIG. 6 illustrates an alternative embodiment of the example operational flow of FIG. 3.

FIG. 7 illustrates an alternative embodiment of the example operational flow of FIG. 3.

FIG. 8 illustrates an alternative embodiment of the example operational flow of FIG. 3.

FIG. 9 illustrates an alternative embodiment of the example operational flow of FIG. 3.

With reference now to FIG. 10, shown is a partial view of an example computer program product that includes a computer program for executing a computer process on a computing device related to computational user-health testing, which may serve as a context for introducing one or more processes and/or devices described herein.

With reference now to FIG. 11, shown is an example device in which embodiments may be implemented related to computational user-health testing, which may serve as a context for introducing one or more processes and/or devices described herein.

The use of the same symbols in different drawings typically indicates similar or identical items.

### DETAILED DESCRIPTION

FIG. 1 illustrates an example system 100 in which embodiments may be implemented. The system 100 includes a device 102. The device 102 may contain, for example, an application 106 and a structurally distinct user-health test unit 104. In some embodiments the user-health test unit 104 may be structurally distinct from the device 102 on which the application 106 is operable. In such embodiments the user-health test unit 104 may be implemented, for example, on external device 160. Through interaction with application 106, user 110 may generate user data 116 that may be obtained by device 102 and/or user-health test unit 104. The application 106 may include, for example, a game 144, a communication application 150, a security application 152, and/or a productivity application 154.

The device 102 may optionally include a data capture module 112, a data detection module 114, a user input device 146, and/or a user monitoring device 148. The user-health test unit 104 may include an alertness or attention test module 118, a memory test module 120, a speech test module 122, a calculation test module 124, a neglect or construction test module 126, a task sequencing test module 128, a visual field test module 130, a pupillary reflex or eye movement test module 132, a face pattern test module 134, a hearing test module 136, a voice test module 138, a motor skill test module 140, or a body movement test module 142.

In FIG. 1, the device 102 is illustrated as possibly being included within a system 100. Of course, virtually any kind of computing device may be used to implement the user-health test unit 104, such as, for example, a workstation, a desktop computer, a networked computer, a collection of servers and/or databases, or a tablet PC.

Additionally, not all of the user-health test unit 104 need be implemented on a single computing device. For example, the user-health test unit 104 and/or application 106 may be implemented and/or operable on a remote computer, while the user interface 108 and/or user data 116 are implemented and/or stored on a local computer. Further, aspects of the user-health test unit 104 may be implemented in different combinations and implementations than that shown in FIG. 1. For example, functionality of the user interface 108 may be incorporated into the user-health test unit 104. The user-health test unit 104 may perform simple data relay functions and/or complex data analysis, including, for example, fuzzy logic and/or traditional logic steps. Further, many methods of searching databases known in the art may be used, including, for example, unsupervised pattern discovery methods, coincidence detection methods, and/or entity relationship modeling. In some embodiments, the user-health test unit 104 may process user data 116 according to health profiles available as updates through a network.

The user data 116 may be stored in virtually any type of memory that is able to store and/or provide access to information in, for example, a one-to-many, many-to-one, and/or many-to-many relationship. Such a memory may include, for example, a relational database and/or an object-oriented database, examples of which are provided in more detail herein.

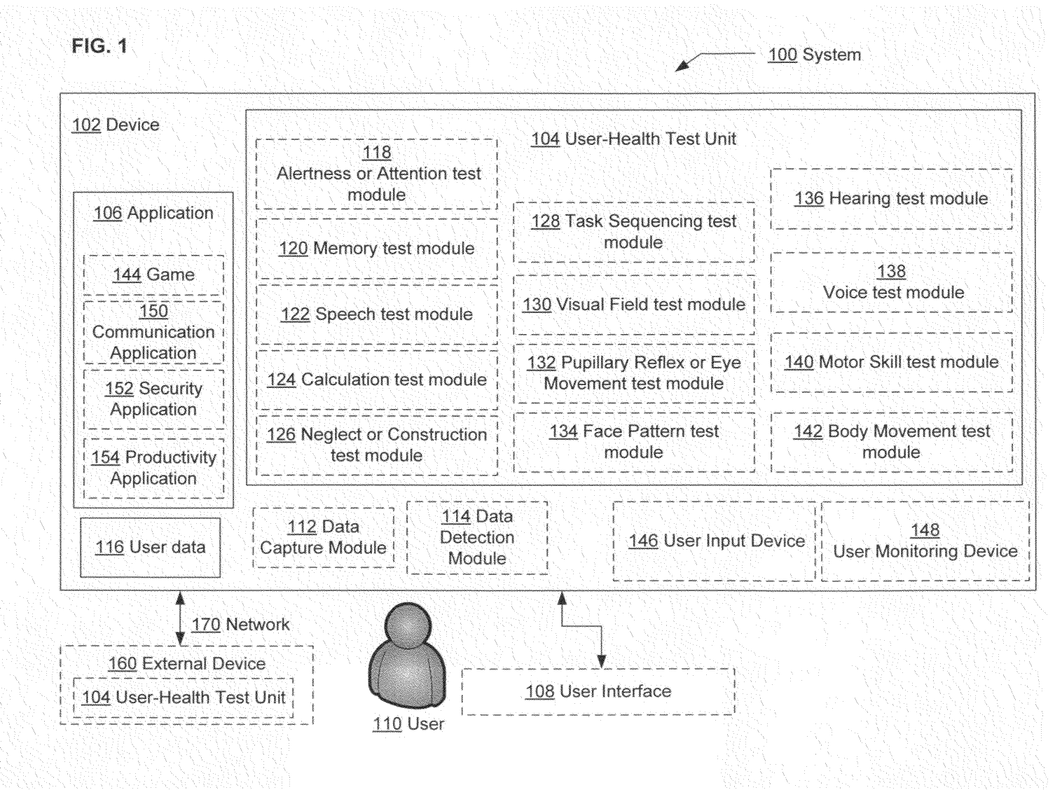

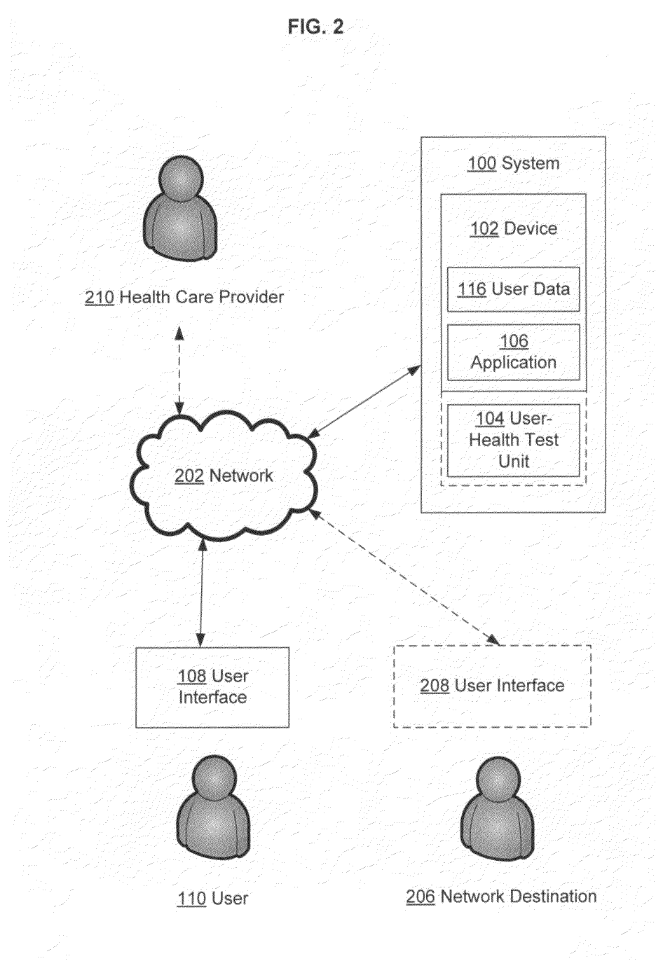

FIG. 2 illustrates certain alternative embodiments of the system 100 of FIG. 1. In FIG. 2, the user 110 uses the user interface 108 to interact through a network 202 with the application 106 operable on a device 102. A user-health test unit 104 may be implemented on the device 102 or on another device within the system 100 but separate from the device 102. The device 102 may be in communication over a network 202 with a network destination 206 and/or healthcare provider 210, which may interact with the device 102 and/or the user-health test unit 104 through, for example, a user interface 208. Of course, it should be understood that there may be many users other than the specifically-illustrated user 110, for example, each with access to a local instance of the application 106.

In this way, the user 110, who may be using a device that is connected through a network 202 with the system 100 (e.g., in an office, outdoors and/or in a public environment), may generate user data 116 as if the user 110 were interacting locally with the device 102 on which the application 106 is locally operable.

As referenced herein, the user-health test unit 104 may be used to perform various data querying and/or recall techniques with respect to the user data 116, in order to present an output of the user-health test function at least partly based on the user data. For example, where the user data 116 is organized, keyed to, and/or otherwise accessible using one or more reference health condition attributes or profiles, various Boolean, statistical, and/or semi-boolean searching techniques may be performed to match user data 116 with reference health condition data, attributes, or profiles.

Many examples of databases and database structures may be used in connection with the user-health test unit 104. Such examples include hierarchical models (in which data is organized in a tree and/or parent-child node structure), network models (based on set theory, and in which multi-parent structures per child node are supported), or object/relational models (combining the relational model with the object-oriented model).

Still other examples include various types of eXtensible Mark-up Language (XML) databases. For example, a database may be included that holds data in some format other than XML, but that is associated with an XML interface for accessing the database using XML. As another example, a database may store XML data directly. Additionally, or alternatively, virtually any semi-structured database may be used, so that context may be provided to/associated with stored data elements (either encoded with the data elements, or encoded externally to the data elements), so that data storage and/or access may be facilitated.

Such databases, and/or other memory storage techniques, may be written and/or implemented using various programming or coding languages. For example, object-oriented database management systems may be written in programming languages such as, for example, C++ or Java. Relational and/or object/relational models may make use of database languages, such as, for example, the structured query language (SQL), which may be used, for example, for interactive queries for information and/or for gathering and/or compiling data from the relational database(s).

For example, SQL or SQL-like operations over one or more of reference health condition may be performed, or Boolean operations using a reference health condition may be performed. For example, weighted Boolean operations may be performed in which different weights or priorities are assigned to one or more of the reference health conditions, perhaps relative to one another. For example, a number-weighted, exclusive-OR operation may be performed to request specific weightings of desired (or undesired) health reference data to be included or excluded.

FIG. 3 illustrates an operational flow 300 representing example operations related to computational user-health testing. In FIG. 3 and in following figures that include various examples of operational flows, discussion and explanation may be provided with respect to the above-described system environments of FIGS. 1-2, and/or with respect to other examples and contexts. However, it should be understood that the operational flows may be executed in a number of other environment and contexts, and/or in modified versions of FIGS. 1-2. Also, although the various operational flows are presented in the sequence(s) illustrated, it should be understood that the various operations may be performed in other orders than those which are illustrated, or may be performed concurrently.

After a start operation, operation 310 shows implementing in at least one device at least one user-health test function that is structurally distinct from at least one application whose primary function is different from symptom detection, the at least one application being operable on the at least one device. The user-health test function may be implemented on a device 102 within a system 100. The user-health function may be carried out by a user-health test unit 104 resident on device 102. System 100 may also include application 106 that is operable on device 102, to perform a primary function that is different from symptom detection. For example, a user-health test function may be implemented as a user-health test unit 104 residing on an external device 160, which user-health test unit 104 communicates via a network 170, for example, with the at least one device 102. In this example, the user-health test function may be implemented in the at least one device 102 by virtue of its communication over the network 170, and the user-health test function will be structurally distinct from at least one application 106 operable on the at least one device. The at least one application 106 may reside on the at least one device 102, or the at least one application 106 may not reside on the at least one device 102 but instead be operable on the at least one device 102 from a remote location, for example, through a network or other link.

Operation 320 depicts obtaining user data in response to an interaction between a user and the at least one application. For example, a data detection module 114 and data capture module 112 of the at least one device 102 or associated with the at least one device 102 may obtain user data in response to an interaction between the user and the at least one application. For example, the data detection module 114 and/or data capture module 112 of the at least one device 102 or associated with the at least one device 102 may obtain user input data in response to an interaction between the user and the at least one application.

Operation 330 depicts presenting an output of the at least one user-health test function at least partly based on the user data. For example, the user-health test unit 104 may relay a summary of user data 116 relating to a hand-eye coordination test to a computer connected by a network to the device 102 or to at least one memory.

In this regard, it should be understood that a data signal may first be encoded and/or represented in digital form (i.e., as digital data), prior to the assignment to at least one memory. For example, a digitally-encoded representation of user eye movement data may be stored in a local memory, or may be transmitted for storage in a remote memory.

Thus, an operation may be performed relating either to a local or remote storage of the digital data, or to another type of transmission of the digital data. Of course, as discussed herein, operations also may be performed relating to accessing, querying, processing, recalling, or otherwise obtaining the digital data from a memory, including, for example, receiving a transmission of the digital data from a remote memory. Accordingly, such operation(s) may involve elements including at least an operator (e.g., either human or computer) directing the operation, a transmitting computer, and/or a receiving computer, and should be understood to occur within the United States as long as at least one of these elements resides in the United States.

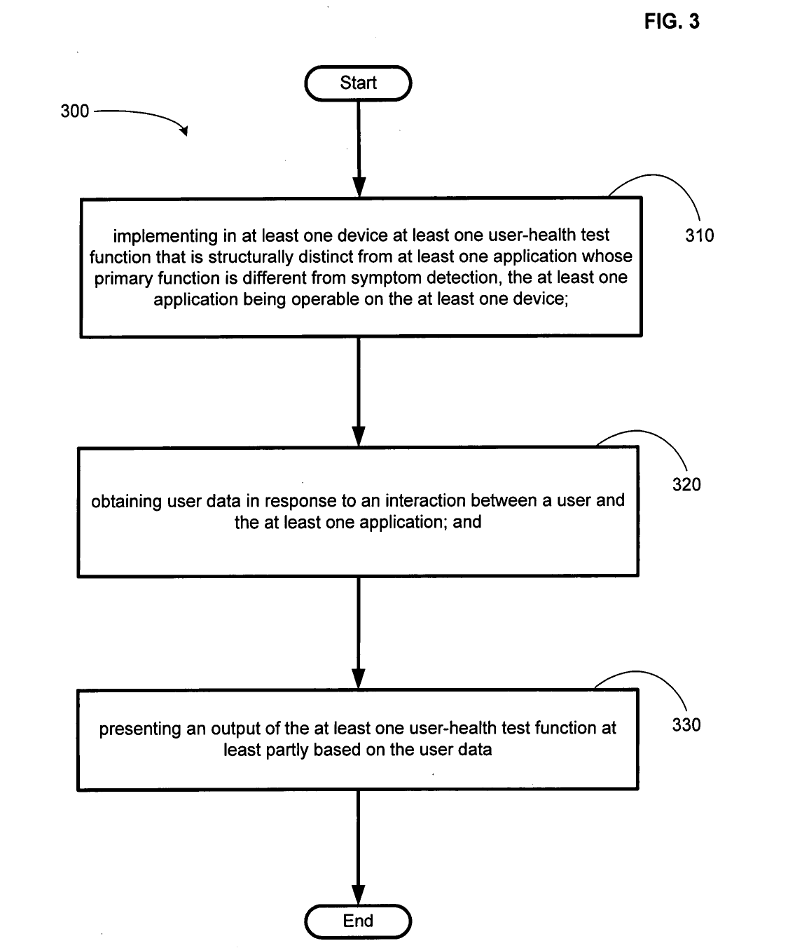

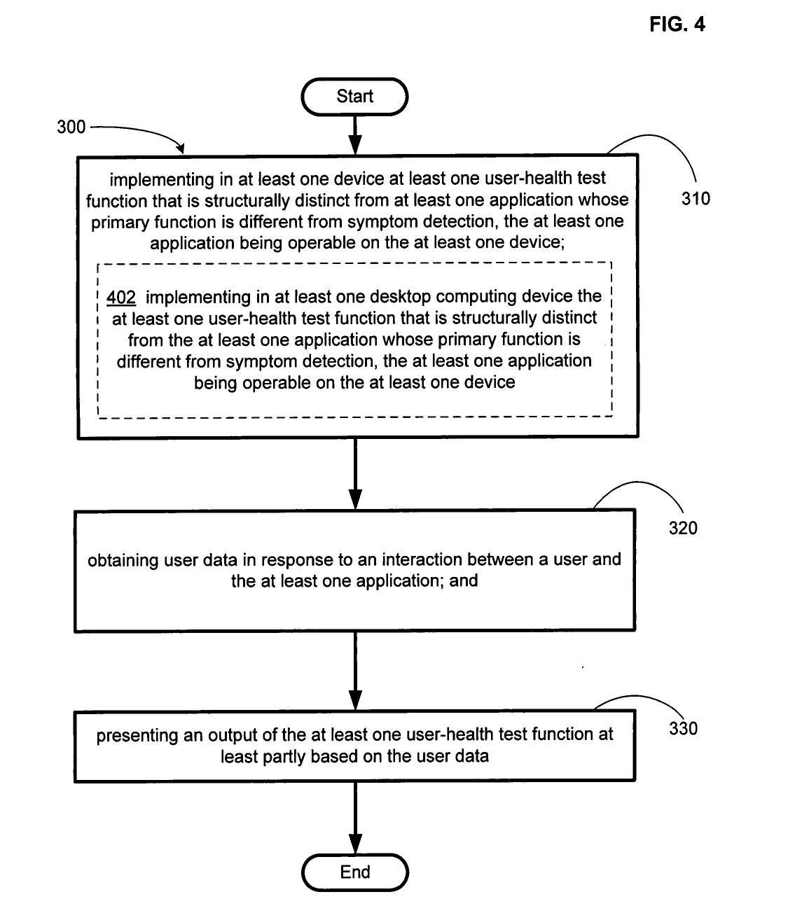

FIG. 4 illustrates alternative embodiments of the example operational flow 300 of FIG. 3. FIG. 4 illustrates example embodiments where the implementing operation 310 may include at least one additional operation. Additional operations may include operation 402.

Operation 402 depicts implementing in at least one desktop computing device the at least one user-health test function that is structurally distinct from the at least one application whose primary function is different from symptom detection, the at least one application being operable on the at least one device. For example, a user-health test function may be implemented in a personal computer of user 110, the user-health test function being structurally distinct from at least one application 106 whose primary function is different from symptom detection, the at least one application 106 being operable on the personal computer of user 110.

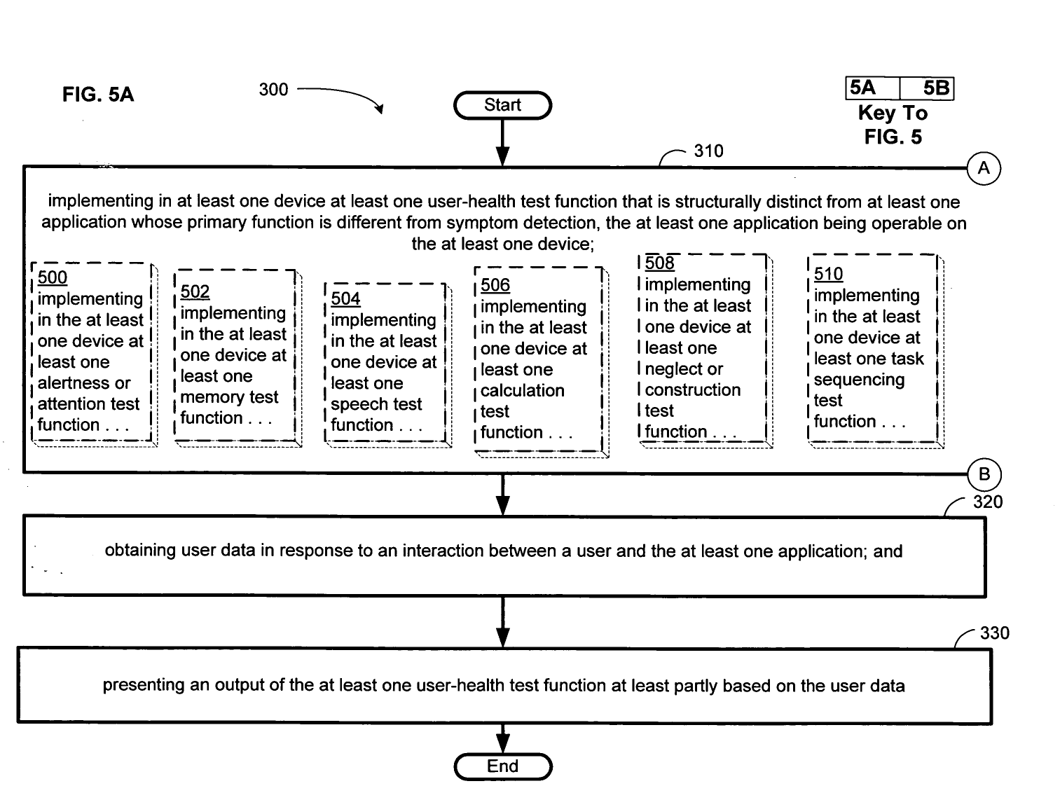

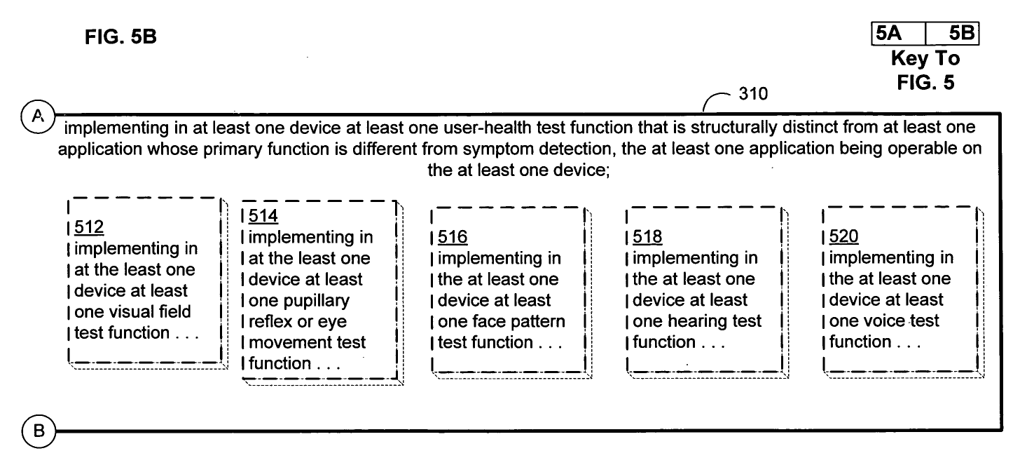

FIG. 5 illustrates alternative embodiments of the example operational flow 300 of FIG. 3. FIG. 5 illustrates example embodiments where the implementing operation 310 may include at least one additional operation. Additional operations may include operation 500, 502, 504, 506, 508, 510, 512, 514, 516, 518, and/or operation 520.

Operation 500 depicts implementing in the at least one device at least one alertness or attention test function that is structurally distinct from the at least one application whose primary function is different from symptom detection, the at least one application being operable on the at least one device. For example, an alertness or attention test module 118 may be implemented in the at least one device 102 that can receive user data 116 from an interaction between the user 110 and the at least one application 106 whose primary function is different from symptom detection, the at least one application 106 being operable on the at least one device 102. Such an alertness or attention test module 118 may receive the user data 116 via a data capture module 112 and/or data detection module 114.

Alertness or attention user attributes are indicators of a user's mental status. An example of an alertness test function may be a measure of reaction time as one objective manifestation. Examples of attention test functions may include ability to focus on simple tasks, ability to spell the word “world” forward and backward, or reciting a numerical sequence forward and backward as objective manifestations of an alertness problem. An alertness or attention test module 118 and/or user-health test unit 104 may require a user to enter a password backward as an alertness test function. Alternatively, a user may be prompted to perform an executive function as a predicate to launching an application such as a word processing program. For example, an alertness test function could be activated by a user command to open a word processing program, requiring performance of, for example, a spelling task as a preliminary step in launching the word processing program. Also, writing ability may be tested by requiring the user to write their name or write a sentence on a device, perhaps with a stylus on a touchscreen.

Reduced level of alertness or attention can indicate the following possible conditions where an acute reduction in alertness or attention is detected: stroke involving the reticular activating system, stroke involving the bilateral or unilateral thalamus, metabolic abnormalities such as hyper or hypoglycemia, toxic effects due to substance overdose (for example, benzodiazepines, or other toxins such as alcohol). Reduced level of alertness and attention can indicate the following possible conditions where a subacute or chronic reduction in alertness or attention is detected: dementia (caused by, for example, Alzheimer's disease, vascular dementia, Parkinson's disease, Huntingdon's disease, Creutzfeldt-Jakob disease, Pick disease, head injury, infection, normal pressure hydrocephalus, brain tumor, exposure to toxin (for example, lead or other heavy metals), metabolic disorders, hormone disorders, hypoxia, drug reactions, drug overuse, drug abuse, encephalitis (caused by, for example, enteroviruses, herpes viruses, or arboviruses), or mood disorders (for example, bipolar disorder, cyclothymic disorder, depression, depressive disorder NOS (not otherwise specified), dysthymic disorder, postpartum depression, or seasonal affective disorder)).

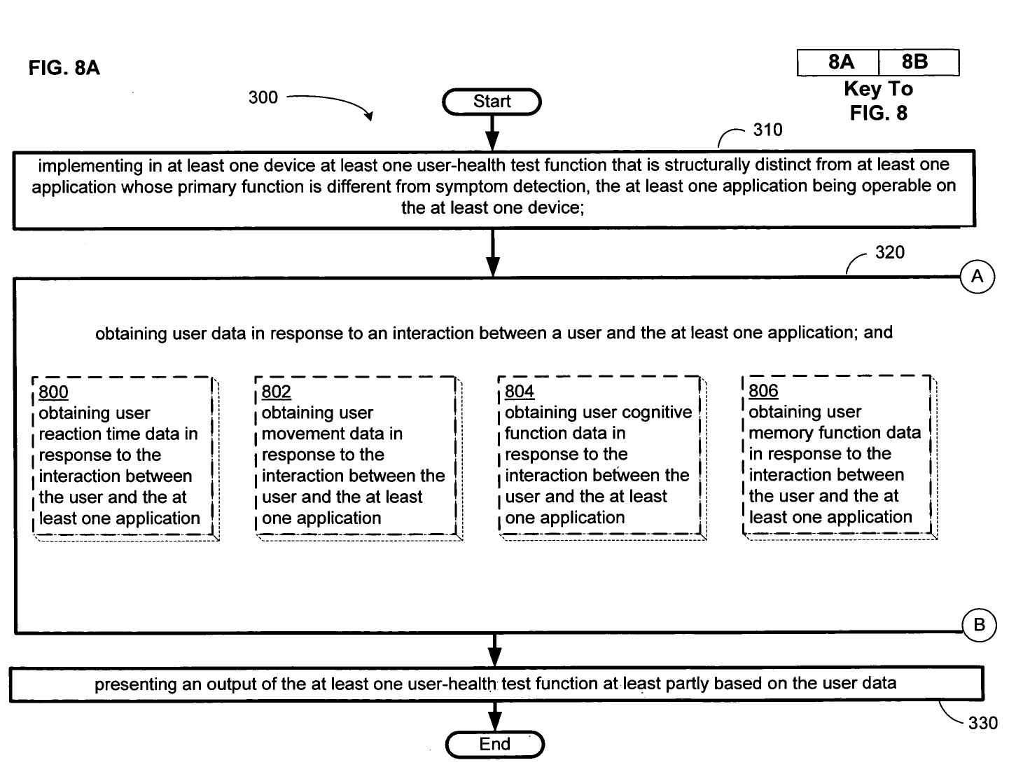

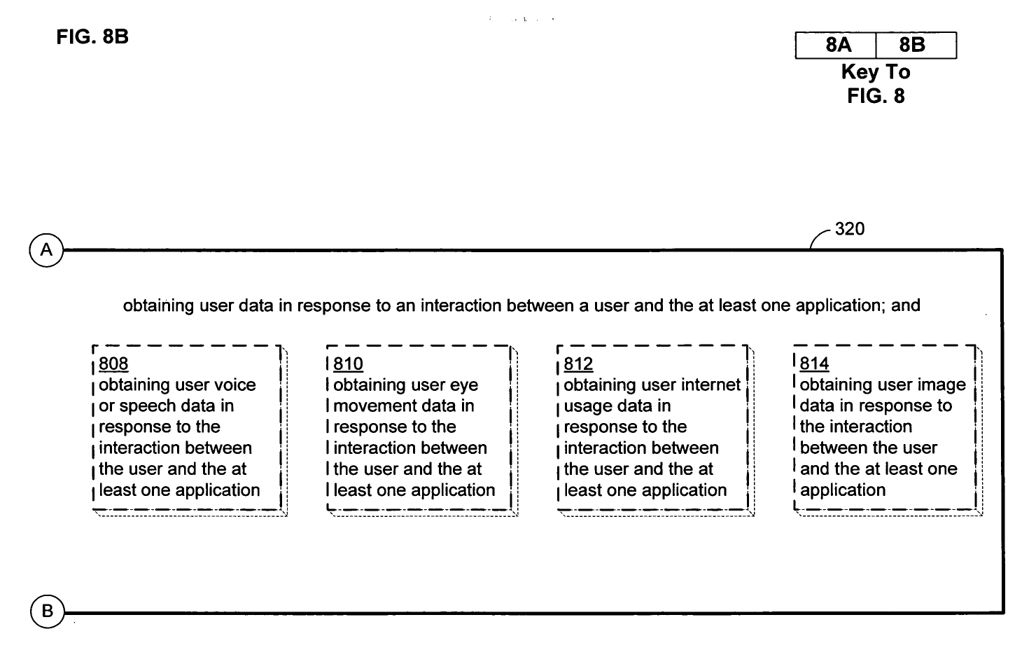

In the context of the above alertness or attention test function, as set forth herein available data arising from the user-health test function are one or more of various types of user data described in FIG. 8 and its supporting text. A reduced level of alertness or attention may indicate certain of the possible conditions discussed above. One skilled in the art can establish or determine parameters or values relating to the one or more types of user data indicative of reduced alertness or attention, or the one or more types of user data indicative of a likely condition associated with reduced alertness or attention. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 502 depicts implementing in the at least one device at least one memory test function that is structurally distinct from the at least one application whose primary function is different from symptom detection, the at least one application being operable on the at least one device. For example, a memory test module 120 may be implemented in the at least one device 102 that can receive user data 116 from an interaction between the user 110 and the at least one application 106 whose primary function is different from symptom detection, the at least one application 106 being operable on the at least one device 102. Such a memory test module 120 may receive the user data 116 via a data capture module 112 or data detection module 114.

A user's memory attributes are indicators of a user's mental status. An example of a memory test function may be a measure of a user's short-term ability to recall items presented, for example, in a story, or after a short period of time. Another example of a memory test function may be a measure of a user's long-term memory, for example their ability to remember basic personal information such as birthdays, place of birth, or names of relatives. Another example of a memory test function may be a memory test module 120 and/or user-health test unit 104 prompting a user to change and enter a password with a specified frequency during internet browser use. A memory test function involving changes to a password that is required to access an internet server can challenge a user's memory according to a fixed or variable schedule.

Difficulty with recall after about 1 to 5 minutes may indicate damage to the limbic memory structures located in the medial temporal lobes and medial diencephalon of the brain, or damage to the formix. Dysfunction of these structures characteristically causes anterograde amnesia, meaning difficulty remembering new facts and events occurring after lesion onset. Reduced short-term memory function can also indicate the following conditions: head injury, Alzheimer's disease, Herpes virus infection, seizure, emotional shock or hysteria, alcohol-related brain damage, barbiturate or heroin use, general anaesthetic effects, electroconvulsive therapy effects, stroke, transient ischemic attack (i.e., a “mini-stroke”), complication of brain surgery. Reduced long-term memory function can indicate the following conditions: Alzheimer's disease, alcohol-related brain damage, complication of brain surgery, depressive pseudodementia, adverse drug reactions (e.g., to benzodiazepines, anti-ulcer drugs, analgesics, anti-hypertensives, diabetes drugs, beta-blockers, anti-Parkinson's disease drugs, anti-emetics, anti-psychotics, or certain drug combinations, such as haloperidol and methyldopa combination therapy), multi-infarct dementia, or head injury.

In the context of the above memory test function, as set forth herein available data arising from the user-health test function are one or more of various types of user data described in FIG. 8 and its supporting text. A reduced level of memory function may indicate certain of the possible conditions discussed above. One skilled in the art can establish or determine parameters or values relating to the one or more types of user data indicative of reduced memory function, or the one or more types of user data indicative of a likely condition associated with reduced memory function. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 504 depicts implementing in the at least one device at least one speech test function that is structurally distinct from the at least one application whose primary function is different from symptom detection, the at least one application being operable on the at least one device. For example, a speech test module 122 may be implemented in the at least one device 102 that can receive user data 116 from an interaction between the user 110 and the at least one application 106 whose primary function is different from symptom detection, the at least one application 106 being operable on the at least one device 102. Such a speech test module 122 may receive the user data 116 via a data capture module 112 or data detection module 114.

User speech attributes are indicators of a user's mental status. An example of a speech test function may be a measure of a user's fluency or ability to produce spontaneous speech, including phrase length, rate of speech, abundance of spontaneous speech, tonal modulation, or whether paraphasic errors (e.g., inappropriately substituted words or syllables), neologisms (e.g., nonexistent words), or errors in grammar are present. Another example of a speech test function is a program that can measure the number of words spoken by a user during a video conference. The number of words per interaction or per unit time could be measured. A marked decrease in the number of words spoken could indicate a speech problem.

Another example of a speech test function may be a measure of a user's comprehension of spoken language, including whether a user 110 can understand simple questions and commands, or grammatical structure. For example, a user 110 could be tested by a speech test module 122 and/or user-health test unit 104 asking the question “Mike was shot by John. Is John dead?” An inappropriate response may indicate a speech center defect. Alternatively a user-health test unit 104 and/or speech test module 122 may require a user to say a code or phrase and repeat it several times. Speech defects may become apparent if the user has difficulty repeating the code or phrase during, for example, a videoconference setup or while using speech recognition software.

Another example of a speech test function may be a measure of a user's ability to name simple everyday objects (e.g., pen, watch, tie) and also more difficult objects (e.g., fingernail, belt buckle, stethoscope). A speech test function may, for example, require the naming of an object prior to or during the interaction of a user 110 with an application 106, as a time-based or event-based checkpoint. For example, a user 110 may be prompted by the user-health test unit 104 and/or the speech test module 122 to say “armadillo” after being shown a picture of an armadillo, prior to or during the user's interaction with, for example, a word processing or email program. A test requiring the naming of parts of objects is often more difficult for users with speech comprehension impairment. Another speech test gauges a user's ability to repeat single words and sentences (e.g., “no if's and's or but's”). A further example of a speech test measures a user's ability to read single words, a brief written passage, or the front page of the newspaper aloud followed by a test for comprehension.

Difficulty with speech or reading/writing ability may indicate, for example, lesions in the dominant (usually left) frontal lobe, including Broca's area (output area); the left temporal and parietal lobes, including Wernicke's area (input area); subcortical white matter and gray matter structures, including thalamus and caudate nucleus; as well as the non-dominant hemisphere. Typical diagnostic conditions may include, for example, stroke, head trauma, dementia, multiple sclerosis, Parkinson's disease, Landau-Kleffner syndrome (a rare syndrome of acquired epileptic aphasia).

In the context of the above speech test function, as set forth herein available data arising from the user-health test function are one or more of various types of user data described in FIG. 8 and its supporting text. A reduced level of speech ability may indicate certain of the possible conditions discussed above. One skilled in the art can establish or determine parameters or values relating to the one or more types of user data indicative of reduced speech ability, or the one or more types of user data indicative of a likely condition associated with reduced speech ability. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 506 depicts implementing in the at least one device at least one calculation test function that is structurally distinct from the at least one application whose primary function is different from symptom detection, the at least one application being operable on the at least one device. For example, a calculation test module 124 may be implemented in the at least one device 102 that can receive user data 116 from an interaction between the user 110 and the at least one application 106 whose primary function is different from symptom detection, the at least one application 106 being operable on the at least one device 102. Such a calculation test module 124 may receive the user data 116 via a data capture module 112 or data detection module 114.

A user's calculation attributes are indicators of a user's mental status. An example of a calculation test function may be a measure of a user's ability to do simple math such as addition or subtraction, for example. A calculation test module 124 and/or user-health test unit 104 may prompt a user 110 to solve an arithmetic problem in the context of interacting with application 106, or alternatively, in the context of using the device in between periods of interacting with the application 106. For example, a user may be prompted to enter the number of items and/or gold pieces collected during a segment of gameplay in the context of playing a game. In this and other contexts, user interaction with a device's operating system or other system function may also constitute user interaction with an application 106. Difficulty in completing calculation tests may be indicative of stroke (e.g., embolic, thrombotic, or due to vasculitis), dominant parietal lesion, or brain tumor (e.g., glioma or meningioma). When a calculation ability deficiency is found with defects in user ability to distinguish right and left body parts (right-left confusion), ability to name and identify each finger (finger agnosia), and ability to write their name and a sentence, Gerstman's syndrome, a lesion in the dominant parietal lobe of the brain, may be present.

In the context of the above calculation test function, as set forth herein available data arising from the user-health test function are one or more of various types of user data described in FIG. 8 and its supporting text. A reduced level of calculation ability may indicate certain of the possible conditions discussed above. One skilled in the art can establish or determine parameters or values relating to the one or more types of user data indicative of reduced calculation ability, or the one or more types of user data indicative of a likely condition associated with reduced calculation ability. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 508 depicts implementing in the at least one device at least one neglect or construction test function that is structurally distinct from the at least one application whose primary function is different from symptom detection, the at least one application being operable on the at least one device. For example, a neglect or construction test module 126 may be implemented in the at least one device 102 that can receive user data 116 from an interaction between the user 110 and the at least one application 106 whose primary function is different from symptom detection, the at least one application 106 being operable on the at least one device 102. Such a neglect or construction test module 126 may receive the user data 116 via a data capture module 112 or data detection module 114.

Neglect or construction user attributes are indicators of a user's mental status. Neglect may include a neurological condition involving a deficit in attention to an area of space, often one side of the body or the other. A construction defect may include a deficit in a user's ability to draw complex figures or manipulate blocks or other objects in space as a result of neglect or other visuospatial impairment.

Hemineglect may include an abnormality in attention to one side of the universe that is not due to a primary sensory or motor disturbance. In sensory neglect, users ignore visual, somatosensory, or auditory stimuli on the affected side, despite intact primary sensation. This can often be demonstrated by testing for extinction on double simultaneous stimulation. Thus, a neglect or construction test module 126 and/or user-health test unit 104 may present a stimulus on one or both sides of a display for a user 110 to click on. A user with hemineglect may detect the stimulus on the affected side when presented alone, but when stimuli are presented simultaneously on both sides, only the stimulus on the unaffected side may be detected. In motor neglect, normal strength may be present, however, the user often does not move the affected limb unless attention is strongly directed toward it.

An example of a neglect test function may be a measure of a user's awareness of events occurring on one side of the user or the other. A user could be asked, “Do you see anything on the left side of the screen?” Users with anosognosia (i.e., unawareness of a disability) may be strikingly unaware of severe deficits on the affected side. For example, some people with acute stroke who are completely paralyzed on the left side believe there is nothing wrong and may even be perplexed about why they are in the hospital. Alternatively, a neglect or construction test module 126 and/or user-health test unit 104 may present a drawing task to a user in the context of an application 106 that involves similar activities. A construction test involves prompting a user to draw complex figures or to manipulate objects in space. Difficulty in completing such a test may be a result of neglect or other visuospatial impairment.

Another neglect test function is a test of a user's ability to acknowledge a series of objects on a display that span a center point on the display. For example, a user may be prompted to click on each of 5 hash marks present in a horizontal line across the midline of a display. If the user has a neglect problem, she may only detect and accordingly click on the hash marks on one side of the display, neglecting the others.

Hemineglect is most common in lesions of the right (nondominant) parietal lobe, causing users to neglect the left side. Left-sided neglect can also occasionally be seen in right frontal lesions, right thalamic or basal ganglia lesions, and, rarely, in lesions of the right midbrain. Hemineglect or difficulty with construction tasks may be indicative of stroke (e.g., embolic, thrombotic, or due to vasculitis), or brain tumor (e.g., glioma or meningioma).

In the context of the above neglect or construction test function, as set forth herein available data arising from the user-health test function are one or more of various types of user data described in FIG. 8 and its supporting text. A change in neglect or in construction ability may indicate certain of the possible conditions discussed above. One skilled in the art can establish or determine parameters or values relating to the one or more types of user data indicative of a change in neglect or in construction ability, or the one or more types of user data indicative of a likely condition associated with a change in neglect or in construction ability. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 510 depicts implementing in the at least one device at least one task sequencing test function that is structurally distinct from the at least one application whose primary function is different from symptom detection, the at least one application being operable on the at least one device. For example, a task sequencing test module 128 may be implemented in the at least one device 102 that can receive user data 116 from an interaction between the user 110 and the at least one application 106 whose primary function is different from symptom detection, the at least one application 106 being operable on the at least one device 102. Such a task sequencing test module 128 may receive the user data 116 via a data capture module 112 or data detection module 114.

A user's task sequencing attributes are indicators of a user's mental status. An example of a task sequencing test function may be a measure of a user's perseveration. For example, a task sequencing test module 128 and/or user-health test unit 104 may ask a user to continue drawing a silhouette pattern of alternating triangles and squares (i.e., a written alternating sequencing task) for a time period. In users with perseveration problems, the user may get stuck on one shape and keep drawing triangles. Another common finding is motor impersistence, a form of distractibility in which users only briefly sustain a motor action in response to a command such as “raise your arms” or “Look to the right.” Ability to suppress inappropriate behaviors can be tested by the auditory “Go-No-Go” test, in which the user moves a finger in response to one sound, but must keep it still in response to two sounds. Alternatively, a task sequencing test module 128 and/or user-health test unit 104 may prompt a user to perform a multi-step function in the context of an application 106, for example. For example, a game may prompt a user to enter a character's name, equip an item from an inventory, an click on a certain direction of travel, in that order. Difficulty completing this task may indicate, for example, a frontal lobe defect associated with dementia.

Decreased ability to perform sequencing tasks may be indicative of stroke (e.g., embolic, thrombotic, or due to vasculitis), brain tumor (e.g., glioma or meningioma), or dementia (caused by, for example, Alzheimer's disease, vascular dementia, Parkinson's disease, Huntingdon's disease, Creutzfeldt-Jakob disease, Pick disease, head injury, infection (e.g., meningitis, encephalitis, HIV, or syphilis), normal pressure hydrocephalus, brain tumor, exposure to toxin (for example, lead or other heavy metals), metabolic disorders, hormone disorders, hypoxia (caused by, e.g., emphysema, pneumonia, or congestive heart failure), drug reactions (e.g., anti-cholinergic side effects, drug overuse, drug abuse (e.g., cocaine or heroin).

In the context of the above task sequencing test function, as set forth herein available data arising from the user-health test function are one or more of various types of user data described in FIG. 8 and its supporting text. A reduced level of task sequencing ability may indicate certain of the possible conditions discussed above. One skilled in the art can establish or determine parameters or values relating to the one or more types of user data indicative of reduced task sequencing ability, or the one or more types of user data indicative of a likely condition associated with reduced task sequencing ability. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 512 depicts implementing in the at least one device at least one visual field test function that is structurally distinct from the at least one application whose primary function is different from symptom detection, the at least one application being operable on the at least one device. For example, a visual field test module 130 may be implemented in the at least one device 102 that can receive user data 116 from an interaction between the user 110 and the at least one application 106 whose primary function is different from symptom detection, the at least one application 106 being operable on the at least one device 102. Such a visual field test module 130 may receive the user data 116 via a data capture module 112 or data detection module 114.

An example of a visual field test function may be a measure of a user's gross visual acuity, for example using a Snellen eye chart or visual equivalent on a display. Alternatively, a campimeter may be used to conduct a visual field test. A visual field test module 130 and/or user-health test unit 104 can prompt a user to activate a portion of a display when the user can detect an object entering their field of view from a peripheral location relative to a fixed point of focus, either with both eyes or with one eye covered at a time. Such testing could be done in the context of, for example, new email alerts that require clicking and that appear in various locations on a display. Based upon the location of decreased visual field, the defect can be localized, for example in a quadrant system. A pre-chiasmatic lesion results in ipsilateral eye blindness. A chiasmatic lesion can result in bi-temporal hemianopsia (i.e., tunnel vision). Post-chiasmatic lesions proximal to the geniculate ganglion can result in left or right homonymous hemianopsia. Lesions distal to the geniculate ganglion can result in upper or lower homonymous quadrantanopsia.

Visual field defects may indicate optic nerve conditions such as pre-chiasmatic lesions, which include fractures of the sphenoid bone (e.g., transecting the optic nerve), retinal tumors, or masses compressing the optic nerve. Such conditions may result in unilateral blindness and unilaterally unreactive pupil (although the pupil may react to light applied to the contralateral eye). Bi-temporal hemianopsia can be caused by glaucoma, pituitary adenoma, craniopharyngioma or saccular Berry aneurysm at the optic chiasm. Post-chiasmatic lesions are associated with homonymous hemianopsia or quadrantanopsia depending on the location of the lesion.

In the context of the above visual field test function, as set forth herein available data arising from the user-health test function are one or more of various types of user data described in FIG. 8 and its supporting text. A reduced visual field may indicate certain of the possible conditions discussed above. One skilled in the art can establish or determine parameters or values relating to the one or more types of user data indicative of reduced visual field, or the one or more types of user data indicative of a likely condition associated with reduced visual field. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 514 depicts implementing in the at least one device at least one pupillary reflex or eye movement test function that is structurally distinct from the at least one application whose primary function is different from symptom detection, the at least one application being operable on the at least one device. For example, a pupillary reflex or eye movement test module 132 may be implemented in the at least one device 102 that can receive user data 116 from an interaction between the user 110 and the at least one application 106 whose primary function is different from symptom detection, the at least one application 106 being operable on the at least one device 102. Such a pupillary reflex or eye movement test module 132 may receive the user data 116 via a data capture module 112 or data detection module 114.

An example of a pupillary reflex test function may be a measure of a user's pupils when exposed to light or objects at various distances. A pupillary reflex or eye movement test module 132 and/or user-health test unit 104 may assess the size and symmetry of a user's pupils before and after a stimulus, such as light or focal point. Anisocoria (i.e., unequal pupils) of up to 0.5 mm is fairly common, and is benign provided pupillary reaction to light is normal. Pupillary reflex can be tested in a darkened room by shining light in one pupil and observing any constriction of the ipsilateral pupil (direct reflex) or the contralateral pupil (contralateral reflex). If abnormality is found with light reaction, pupillary accommodation can be tested by having the user focus on an object at a distance, then focus on the object at about 10 cm from the nose. Pupils should converge and constrict at close focus.

Pupillary abnormalities may be a result of either optic nerve or oculomotor nerve lesions. An optic nerve lesion (e.g., blind eye) will not react to direct light and will not elicit a consensual pupillary constriction, but will constrict if light is shown in the opposite eye. A Horner's syndrome lesion (sympathetic chain lesion) can also present as a pupillary abnormality. In Horner's syndrome, the affected pupil is smaller but constricts to both light and near vision and may be associated with ptosis and anhydrosis. In an oculomotor nerve lesion, the affected pupil is fixed and dilated and may be associated with ptosis and lateral deviation (due to unopposed action of the abducens nerve). Small pupils that do not react to light but do constrict with near vision (i.e., accommodate but do not react to light) can be seen in central nervous system syphilis (“Argyll Robertson pupil”).

Pupillary reflex deficiencies may indicate damage to the oculomotor nerve in basilar skull fracture or uncal herniation as a result of increased intracranial pressure. Masses or tumors in the cavernous sinus, syphilis, or aneurysm may also lead to compression of the oculomotor nerve. Injury to the oculomotor nerve may result in ptosis, inferolateral displacement of the ipsilateral eye (which can present as diplopia or strabismus), or mydriasis.

An example of an eye movement test function may be a pupillary reflex or eye movement test module 132 and/or user-health test unit 104 measurement of a user's ability to follow a target on a display with her eyes throughout a 360° range. Such testing may be done in the context of a user playing a game or participating in a videoconference. In such examples, user data 116 may be obtained through a camera in place as a user monitoring device 148 that can monitor the eye movements of the user during interaction with the application 106.

Testing of the trochlear nerve or the abducens nerve for damage may involve measurement of extraocular movements. The trochlear nerve performs intorsion, depression, and abduction of the eye. A trochlear nerve lesion may present as extorsion of the ipsilateral eye and worsened diplopia when looking down. Damage to the abducens nerve may result in a decreased ability to abduct the eye.

Abnormalities in eye movement may indicate fracture of the sphenoid wing, intracranial hemorrhage, neoplasm, or aneurysm. Such insults may present as extorsion of the ipsilateral eye. Individuals with this condition complain of worsened diplopia with attempted downgaze, but improved diplopia with head tilted to the contralateral side. Injury to the abducens nerve may be caused by aneurysm, a mass in the cavernous sinus, or a fracture of the skull base. Such insults may result in extraocular palsy defined by medial deviation of the ipsilateral eye. Users with this condition may present with diplopia that improves when the contralateral eye is abducted.

Nystagmus is a rapid involuntary rhythmic eye movement, with the eyes moving quickly in one direction (quick phase), and then slowly in the other direction (slow phase). The direction of nystagmus is defined by the direction of its quick phase (e.g., right nystagmus is due to a right-moving quick phase). Nystagmus may occur in the vertical or horizontal directions, or in a semicircular movement. Terminology includes downbeat nystagmus, upbeat nystagmus, seesaw nystagmus, periodic alternating nystagmus, and pendular nystagmus. There are other similar alterations in periodic eye movements (saccadic oscillations) such as opsoclonus or ocular flutter. One can think of nystagmus as the combination of a slow adjusting eye movement (slow phase) as would be seen with the vestibulo-ocular reflex, followed by a quick saccade (quick phase) when the eye has reached the limit of its rotation.

In medicine, the clinical importance of nystagmus is that it indicates that the user's spatial sensory system perceives rotation and is rotating the eyes to adjust. Thus it depends on the coordination of activities between two major physiological systems: the vision and the vestibular apparatus (which controls posture and balance). This may be physiological (i.e., normal) or pathological.

Vestibular nystagmus may be central or peripheral. Important differentiating features between central and peripheral nystagmus include the following: peripheral nystagmus is unidirectional with the fast phase opposite the lesion; central nystagmus may be unidirectional or bidirectional; purely vertical or torsional nystagmus suggests a central location; central vestibular nystagmus is not dampened or inhibited by visual fixation; tinnitus or deafness often is present in peripheral vestibular nystagmus, but it usually is absent in central vestibular nystagmus. According to Alexander's law, the nystagmus associated with peripheral lesions becomes more pronounced with gaze toward the side of the fast-beating component; with central nystagmus, the direction of the fast component is directed toward the side of gaze (e.g., left-beating in left gaze, right-beating in right gaze, and up-beating in upgaze).

Downbeat nystagmus is defined as nystagmus with the fast phase beating in a downward direction. The nystagmus usually is of maximal intensity when the eyes are deviated temporally and slightly inferiorly. With the eyes in this position, the nystagmus is directed obliquely downward. In most users, removal of fixation (e.g., by Frenzel goggles) does not influence slow phase velocity to a considerable extent, however, the frequency of saccades may diminish.

The presence of downbeat nystagmus is highly suggestive of disorders of the cranio-cervical junction (e.g., Arnold-Chiari malformation). This condition also may occur with bilateral lesions of the cerebellar flocculus and bilateral lesions of the medial longitudinal fasciculus, which carries optokinetic input from the posterior semicircular canals to the third nerve nuclei. It may also occur when the tone within pathways from the anterior semicircular canals is relatively higher than the tone within the posterior semicircular canals. Under such circumstances, the relatively unopposed neural activity from the anterior semicircular canals causes a slow upward pursuit movement of the eyes with a fast, corrective downward saccade. Additional causes include demyelination (e.g., as a result of multiple sclerosis), microvascular disease with vertebrobasilar insufficiency, brain stem encephalitis, tumors at the foramen magnum (e.g., meningioma, or cerebellar hemangioma), trauma, drugs (e.g., alcohol, lithium, or anti-seizure medications), nutritional imbalances (e.g., Wemicke encephalopathy, parenteral feeding, magnesium deficiency), or heat stroke.

Upbeat nystagmus is defined as nystagmus with the fast phase beating in an upward direction. Daroff and Troost described two distinct types. The first type consists of a large amplitude nystagmus that increases in intensity with upward gaze. This type is suggestive of a lesion of the anterior vermis of the cerebellum. The second type consists of a small amplitude nystagmus that decreases in intensity with upward gaze and increases in intensity with downward gaze. This type is suggestive of lesions of the medulla, including the perihypoglossal nuclei, the adjacent medial vestibular nucleus, and the nucleus intercalatus (structures important in gaze-holding). Upbeat nystagmus may also be an indication of benign paroxysmal positional vertigo.

Torsional (rotary) nystagmus refers to a rotary movement of the globe about its anteroposterior axis. Torsional nystagmus is accentuated on lateral gaze. Most nystagmus resulting from dysfunction of the vestibular system has a torsional component superimposed on a horizontal or vertical nystagmus. This condition occurs with lesions of the anterior and posterior semicircular canals on the same side (e.g., lateral medullary syndrome or Wallenberg syndrome). Lesions of the lateral medulla may produce a torsional nystagmus with the fast phase directed away from the side of the lesion. This type of nystagmus can be accentuated by otolithic stimulation by placing the user on their side with the intact side down (e.g., if the lesion is on the left, the nystagmus is accentuated when the user is placed on his right side).

This condition may occur when the tone within the pathways of the posterior semicircular canals is relatively higher than the tone within the anterior semicircular canals, and it can occur from lesions of the ventral tegmental tract or the brachium conjunctivum, which carry optokinetic input from the anterior semicircular canals to the third nerve nuclei.

Pendular nystagmus is a multivectorial nystagmus (i.e., horizontal, vertical, circular, and elliptical) with an equal velocity in each direction that may reflect brain stem or cerebellar dysfunction. Often, there is marked asymmetry and dissociation between the eyes. The amplitude of the nystagmus may vary in different positions of gaze. Causes of pendular nystagmus may include demyelinating disease, monocular or binocular visual deprivation, oculapalatal myoclonus, internuclear opthalmoplegia, or brain stem or cerebellar dysfunction.

Horizontal nystagmus is a well-recognized finding in patients with a unilateral disease of the cerebral hemispheres, especially with large, posterior lesions. It often is of low amplitude. Such patients show a constant velocity drift of the eyes toward the intact hemisphere with fast saccade directed toward the side of the lesion.

Seesaw nystagmus is a pendular oscillation that consists of elevation and intorsion of one eye and depression and extorsion of the fellow eye that alternates every half cycle. This striking and unusual form of nystagmus may be seen in patients with chiasmal lesions, suggesting loss of the crossed visual inputs from the decussating fibers of the optic nerve at the level of the chiasm as the cause or lesions in the rostral midbrain. This type of nystagmus is not affected by otolithic stimulation. Seesaw nystagmus may also be caused by parasellar lesions or visual loss secondary to retinitis pigmentosa.

Gaze-evoked nystagmus is produced by the attempted maintenance of an extreme eye position. It is the most common form of nystagmus. Gaze-evoked nystagmus is due to a deficient eye position signal in the neural integrator network. Thus, the eyes cannot be maintained at an eccentric orbital position and are pulled back toward primary position by the elastic forces of the orbital fascia. Then, corrective saccade moves the eyes back toward the eccentric position in the orbit.

Gaze-evoked nystagmus may be caused by structural lesions that involve the neural integrator network, which is dispersed between the vestibulocerebellum, the medulla (e.g., the region of the nucleus prepositus hypoglossi and adjacent medial vestibular nucleus “NPH/MVN”), and the interstitial nucleus of Cajal (“INC”). Patients recovering from a gaze palsy go through a period where they are able to gaze in the direction of the previous palsy, but they are unable to sustain gaze in that direction; therefore, the eyes drift slowly back toward primary position followed by a corrective saccade. When this is repeated, a gaze-evoked or gaze-paretic nystagmus results.

Gaze-evoked nystagmus often is encountered in healthy users; in which case, it is called end-point nystagmus. End-point nystagmus usually can be differentiated from gaze-evoked nystagmus caused by disease, in that the former has lower intensity and, more importantly, is not associated with other ocular motor abnormalities. Gaze-evoked nystagmus also may be caused by alcohol or drugs including anti-convulsants (e.g., phenobarbital, phenytoin, or carbamazepine) at therapeutic dosages.

Spasmus nutans is a rare condition with the clinical triad of nystagmus, head nodding, and torticollis. Onset is from age 3-15 months with disappearance by 3 or 4 years. Rarely, it may be present to age 5-6 years. The nystagmus typically consists of small-amplitude, high frequency oscillations and usually is bilateral, but it can be monocular, asymmetric, and variable in different positions of gaze. Spasmus nutans occurs in otherwise healthy children. Chiasmal, suprachiasmal, or third ventricle gliomas may cause a condition that mimics spasmus nutans.

Periodic alternating nystagmus is a conjugate, horizontal jerk nystagmus with the fast phase beating in one direction for a period of approximately 1-2 minutes. The nystagmus has an intervening neutral phase lasting 10-20 seconds; the nystagmus begins to beat in the opposite direction for 1-2 minutes; then the process repeats itself. The mechanism may be disruption of the vestibulo-ocular tracts at the pontomedullary junction. Causes of periodic alternating nystagmus may include Arnold-Chiari malformation, demyelinating disease, spinocerebellar degeneration, lesions of the vestibular nuclei, head trauma, encephalitis, syphilis, posterior fossa tumors, or binocular visual deprivation (e.g., ocular media opacities).

Abducting nystagmus of internuclear opthalmoplegia (“INO”) is nystagmus in the abducting eye contralateral to a medial longitudinal fasciculus (“MLF”) lesion.

In the context of the above pupillary reflex or eye movement test function, as set forth herein available data arising from the user-health test function are one or more of various types of user data described in FIG. 8 and its supporting text. Altered pupillary reflex or eye movement may indicate certain of the possible conditions discussed above. One skilled in the art can establish or determine parameters or values relating to the one or more types of user data indicative of altered pupillary reflex or eye movement, or the one or more types of user data indicative of a likely condition associated with altered pupillary reflex or eye movement. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 516 depicts implementing in the at least one device at least one face pattern test function that is structurally distinct from the at least one application whose primary function is different from symptom detection, the at least one application being operable on the at least one device. For example, a face pattern test module 134 may be implemented in the at least one device 102 that can receive user data 116 from an interaction between the user 110 and the at least one application 106 whose primary function is different from symptom detection, the at least one application 106 being operable on the at least one device 102. Such a face pattern test module 134 may receive the user data 116 via a data capture module 112 or data detection module 114.

An example of a face pattern test function may be a face pattern test module 134 and/or user-health test unit 104 that can compare a user's face while at rest, specifically looking for nasolabial fold flattening or drooping of the corner of the mouth, with the user's face while moving certain facial features. The user may be asked to raise her eyebrows, wrinkle her forehead, show her teeth, puff out her cheeks, or close her eyes tight. Such testing may done via facial pattern recognition software used in conjunction with, for example, a videoconferencing application. Any weakness or asymmetry may indicate a lesion in the facial nerve. In general, a peripheral lesion of the facial nerve may affect the upper and lower face while a central lesion may only affect the lower face.

Abnormalities in facial expression or pattern may indicate a petrous fracture. Peripheral facial nerve injury may also be due to compression, tumor, or aneurysm. Bell's Palsy is thought to be caused by idiopathic inflammation of the facial nerve within the facial canal. A peripheral facial nerve lesion involves muscles of both the upper and lower face and can involve loss of taste sensation from the anterior ⅔ of the tongue (via the chorda tympani). A central facial nerve palsy due to tumor or hemorrhage results in sparing of upper and frontal orbicularis occuli due to crossed innervation. Spared ability to raise eyebrows and wrinkle the forehead helps differentiate a peripheral palsy from a central process. This also may indicate stroke or multiple sclerosis.

In the context of the above face pattern test function, as set forth herein available data arising from the user-health test function are one or more of various types of user data described in FIG. 8 and its supporting text. Altered face pattern may indicate certain of the possible conditions discussed above. One skilled in the art can establish or determine parameters or values relating to the one or more types of user data indicative of altered face pattern, or the one or more types of user data indicative of a likely condition associated with altered face pattern. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 518 depicts implementing in the at least one device at least one hearing test function that is structurally distinct from the at least one application whose primary function is different from symptom detection, the at least one application being operable on the at least one device. For example, a hearing test module 136 may be implemented in the at least one device 102 that can receive user data 116 from an interaction between the user 110 and the at least one application 106 whose primary function is different from symptom detection, the at least one application 106 being operable on the at least one device 102. Such a hearing test module 136 may receive the user data 116 via a data capture module 112 or data detection module 114.

An example of a hearing test function may be a hearing test module 136 and/or user-health test unit 104 conducting a gross hearing assessment of a user's ability to hear sounds. This can be done by simply presenting sounds to the user or determining if the user can hear sounds presented to each of the ears. For example, a hearing test module 136 and/or user-health test unit 104 may vary volume settings or sound frequency on a user's device 102 or within an application 106 over time to test user hearing. Alternatively, a hearing test module 136 and/or user-health test unit 104 in a mobile phone device may carry out various hearing test functions.

Petrous fractures that involve the vestibulocochlear nerve may result in hearing loss, vertigo, or nystagmus (frequently positional) immediately after the injury. Severe middle ear infection can cause similar symptoms but have a more gradual onset. Acoustic neuroma is associated with gradual ipsilateral hearing loss. Due to the close proximity of the vestibulocochlear nerve with the facial nerve, acoustic neuromas often present with involvement of the facial nerve. Neurofibromatosis type II is associated with bilateral acoustic neuromas. Vertigo may be associated with anything that compresses the vestibulocochlear nerve including vascular abnormalities, inflammation, or neoplasm.

In the context of the above hearing test function, as set forth herein available data arising from the user-health test function are one or more of various types of user data described in FIG. 8 and its supporting text. Reduced hearing function may indicate certain of the possible conditions discussed above. One skilled in the art can establish or determine parameters or values relating to the one or more types of user data indicative of reduced hearing function, or the one or more types of user data indicative of a likely condition associated with reduced hearing function. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 520 depicts implementing in the at least one device at least one voice test function that is structurally distinct from the at least one application whose primary function is different from symptom detection, the at least one application being operable on the at least one device. For example, a voice test module 138 may be implemented in the at least one device 102 that can receive user data 116 from an interaction between the user 110 and the at least one application 106 whose primary function is different from symptom detection, the at least one application 106 being operable on the at least one device 102. Such a voice test module 138 may receive the user data 116 via a data capture module 112 or data detection module 114.

An example of a voice test function may be a measure of symmetrical elevation of the palate when the user says “aah,” or a test of the gag reflex. In an ipsilateral lesion of the vagus nerve, the uvula deviates towards the affected side. As a result of its innervation (through the recurrent laryngeal nerve) to the vocal cords, hoarseness may develop as a symptom of vagus nerve injury. A voice test module 138 and/or user-health test unit 104 may monitor user voice frequency or volume data during, for example, gaming, videoconferencing, speech recognition software use, or mobile phone use. Injury to the recurrent laryngeal nerve can occur with lesions in the neck or apical chest. The most common lesions are tumors in the neck or apical chest. Cancers may include lung cancer, esophageal cancer, or squamous cell cancer.

Other voice test functions may involve first observing the tongue (while in floor of mouth) for fasciculations. If present, fasciculations may indicate peripheral hypoglossal nerve dysfunction. Next, the user may be prompted to protrude the tongue and move it in all directions. When protruded, the tongue will deviate toward the side of a lesion (as the unaffected muscles push the tongue more than the weaker side). Gross symptoms of pathology may result in garbled sound in speech (as if there were marbles in the user's mouth). Damage to the hypoglossal nerve affecting voice/speech may indicate neoplasm, aneurysm, or other external compression, and may result in protrusion of the tongue away from side of the lesion for an upper motor neuron process and toward the side of the lesion for a lower motor neuron process. Accordingly, a voice test module 138 and/or user-health test unit 104 may assess a user's ability to make simple sounds or to say words, for example, consistently with an established voice pattern for the user.

In the context of the above voice test function, as set forth herein available data arising from the user-health test function are one or more of various types of user data described in FIG. 8 and its supporting text. Altered voice function may indicate certain of the possible conditions discussed above. One skilled in the art can establish or determine parameters or values relating to the one or more types of user data indicative of altered voice function, or the one or more types of user data indicative of a likely condition associated with altered voice function. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

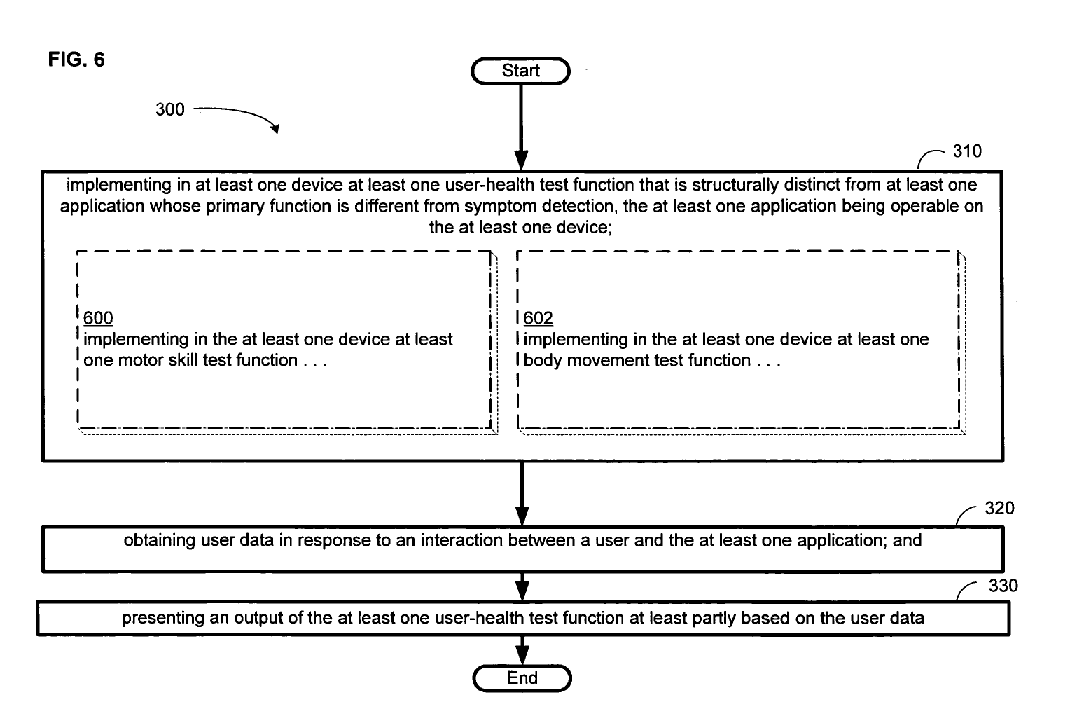

FIG. 6 illustrates alternative embodiments of the example operational flow 300 of FIG. 3. FIG. 6 illustrates example embodiments where the implementing operation 310 may include at least one additional operation. Additional operations may include operation 600 and/or operation 602.

Operation 600 depicts implementing in the at least one device at least one motor skill test function that is structurally distinct from the at least one application whose primary function is different from symptom detection, the at least one application being operable on the at least one device. For example, a motor skill test module 140 may be implemented in the at least one device 102 that can receive user data 116 from an interaction between the user 110 and the at least one application 106 whose primary function is different from symptom detection, the at least one application 106 being operable on the at least one device 102. Such a motor skill test module 140 may receive the user data 116 via a data capture module 112 or data detection module 114.

An example of a motor skill test function may be a measure of a user's ability to perform a physical task, or a measure of tremor in a body part (i.e., a rhythmic, involuntary, or oscillating movement of a body part occurring in isolation or as part of a clinical syndrome). A motor skill test module 140 and/or user-health test unit 104 may measure, for example, a user's ability to traverse a path on a display in straight line with a pointing device, to type a certain sequence of characters without error, or to type a certain number of characters without repetition. For example, a wobbling cursor on a display may indicate ataxia in the user, or a wobbling cursor while the user is asked to maintain the cursor on a fixed point on a display may indicate early Parkinson's disease symptoms. Alternatively, a user may be prompted to switch tasks, for example, to alternately type some characters using a keyboard and click on some target with a mouse. If a user has a motor skill deficiency, she may have difficulty stopping one task and starting the other task.

In clinical practice, characterization of tremor is important for etiologic consideration and treatment. Common types of tremor include resting tremor, postural tremor, action or kinetic tremor, task-specific tremor, or intention or terminal tremor. Resting tremor occurs when a body part is at complete rest against gravity. Tremor amplitude tends to decrease with voluntary activity. Causes of resting tremor may include Parkinson's disease, Parkinson-plus syndromes (e.g., multiple system atrophy, progressive supranuclear palsy, or corticobasal degeneration), Wilson's disease, drug-induced Parkinsonism (e.g., neuroleptics, Reglan, or phenthiazines), or long-standing essential tremor.

Postural tremor occurs during maintenance of a position against gravity and increases with action. Action or kinetic tremor occurs during voluntary movement. Examples of postural and action tremors may include essential tremor (primarily postural), metabolic disorders (e.g., thyrotoxicosis, pheochromocytoma, or hypoglycemia), drug-induced parkinsonism (e.g., lithium, amiodarone, or beta-adrenergic agonists), toxins (e.g., alcohol withdrawal, heavy metals), neuropathic tremor (e.g., neuropathy).

Task-specific tremor emerges during specific activity. An example of this type is primary writing tremor. Intention or terminal tremor manifests as a marked increase in tremor amplitude during a terminal portion of targeted movement. Examples of intention tremor include cerebellar tremor and multiple sclerosis tremor.

In the context of the above motor skill test function, as set forth herein available data arising from the user-health test function are one or more of various types of user data described in FIG. 8 and its supporting text. Altered motor skill function may indicate certain of the possible conditions discussed above. One skilled in the art can establish or determine parameters or values relating to the one or more types of user data indicative of altered motor skill function, or the one or more types of user data indicative of a likely condition associated with altered motor skill function. Parameters or values can be set by one skilled in the art based on knowledge, direct experience, or using available resources such as websites, textbooks, journal articles, or the like. Examples of relevant websites can be found in the online Merck Manual at http://www.merck.com/mmhe/sec06/ch077/ch077c.html#tb077_{—}1; and at http://www.jeffmann.net/NeuroGuidemaps/tremor.html. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

Operation 602 depicts implementing in the at least one device at least one body movement test function that is structurally distinct from the at least one application whose primary function is different from symptom detection, the at least one application being operable on the at least one device. For example, a body movement test module 142 may be implemented in the at least one device 102 that can receive user data 116 from an interaction between the user 110 and the at least one application 106 whose primary function is different from symptom detection, the at least one application 106 being operable on the at least one device 102. Such a body movement test module 142 may receive the user data 116 via a data capture module 112 or data detection module 114.

An example of a body movement test function may be first observing the user for atrophy or fasciculation in the trapezius muscles, shoulder drooping, or displacement of the scapula. A body movement test module 142 and/or user-health test unit 104 may then instruct the user to turn the head and shrug shoulders against resistance. Weakness in turning the head in one direction may indicate a problem in the contralateral spinal accessory nerve, while weakness in shoulder shrug may indicate an ipsilateral spinal accessory nerve lesion. Ipsilateral paralysis of the sternocleidomastoid and trapezius muscles due to neoplasm, aneurysm, or radical neck surgery also may indicate damage to the spinal accessory nerve. A body movement test module 142 and/or user-health test unit 104 may perform gait analysis, for example, in the context of a security system surveillance application involving video monitoring of the user.

Cerebellar disorders can disrupt body coordination or gait while leaving other motor functions relatively intact. The term ataxia is often used to describe the abnormal movements seen in coordination disorders. In ataxia, there are medium- to large-amplitude involuntary movements with an irregular oscillatory quality superimposed on and interfering with the normal smooth trajectory of movement. Overshoot is also commonly seen as part of ataxic movements and is sometimes referred to as “past pointing” when target-oriented movements are being discussed. Another feature of coordination disorders is dysdiadochokinesia (i.e., abnormal alternating movements). Cerebellar lesions can cause different kinds of coordination problems depending on their location. One important distinction is between truncal ataxia and appendicular ataxia. Appendicular ataxia affects movements of the extremities and is usually caused by lesions of the cerebellar hemispheres and associated pathways. Truncal ataxia affects the proximal musculature, especially that involved in gait stability, and is caused by midline damage to the cerebellar vermis and associated pathways.

Fine movements of the hands and feet also may be tested by a body movement test module 142 and/or user-health test unit 104. Rapid alternating movements, such as wiping one palm alternately with the palm and dorsum of the other hand, may be tested as well. A common test of coordination is the finger-nose-finger test, in which the user is asked to alternately touch their nose and an examiner's finger as quickly as possible. Ataxia may be revealed if the examiner's finger is held at the extreme of the user's reach, and if the examiner's finger is occasionally moved suddenly to a different location. Overshoot may be measured by having the user raise both arms suddenly from their lap to a specified level in the air. In addition, pressure can be applied to the user's outstretched arms and then suddenly released. To test the accuracy of movements in a way that requires very little strength, a user can be prompted to repeatedly touch a line drawn on the crease of the user's thumb with the tip of their forefinger; alternatively, a body movement test module 142 and/or user-health test unit 104 may prompt a user to repeatedly touch an object on a touchscreen display.

Normal performance of motor tasks depends on the integrated functioning of multiple sensory and motor subsystems. These include position sense pathways, lower motor neurons, upper motor neurons, the basal ganglia, and the cerebellum. Thus, in order to convincingly demonstrate that abnormalities are due to a cerebellar lesion, one should first test for normal joint position sense, strength, and reflexes and confirm the absence of involuntary movements caused by basal ganglia lesions. As discussed above, appendicular ataxia is usually caused by lesions of the cerebellar hemispheres and associated pathways, while truncal ataxia is often caused by damage to the midline cerebellar vermis and associated pathways.

Another body movement test is the Romberg test, which may indicate a problem in the vestibular or proprioception system. A user is asked to stand with feet together (touching each other). Then the user is prompted to close their eyes. If a problem is present, the user may begin to sway or fall. With the eyes open, three sensory systems provide input to the cerebellum to maintain truncal stability. These are vision, proprioception, and vestibular sense. If there is a mild lesion in the vestibular or proprioception systems, the user is usually able to compensate with the eyes open. When the user closes their eyes, however, visual input is removed and instability can be brought out. If there is a more severe proprioceptive or vestibular lesion, or if there is a midline cerebellar lesion causing truncal instability, the user will be unable to maintain this position even with their eyes open.

In the context of the above body movement test function, as set forth herein available data arising from the user-health test function are one or more of various types of user data described in FIG. 8 and its supporting text. Altered body movement function may indicate certain of the possible conditions discussed above. One skilled in the art can establish or determine parameters or values relating to the one or more types of user data indicative of altered body movement function, or the one or more types of user data indicative of a likely condition associated with altered body movement function. Parameters or values can be set by one skilled in the art based on knowledge, direct experience, or using available resources such as websites, textbooks, journal articles, or the like. An example of a relevant website can be found in the online Merck Manual at http://www.merck.com/mmhe/sec06/ch077/ch077c.html#tb077_{—}1. Examples of relevant textbooks include Patten, J. P., “Neurological Differential Diagnosis,” Second Ed., Springer-Verlag, London, 2005; Kasper, Braunwald, Fauci, Hauser, Longo, and Jameson, “Harrison's Principles of Internal Medicine,” 16^{th} Ed., McGraw-Hill, New York, 2005; Greenberg, M. S., “Handbook of Neurosurgery,” 6^{th} Ed., Thieme, Lakeland, 2006; and Victor, M., and Ropper, A. H., “Adams and Victor's Principles of Neurology,” 7^{th} Ed., McGraw-Hill, New York, 2001.

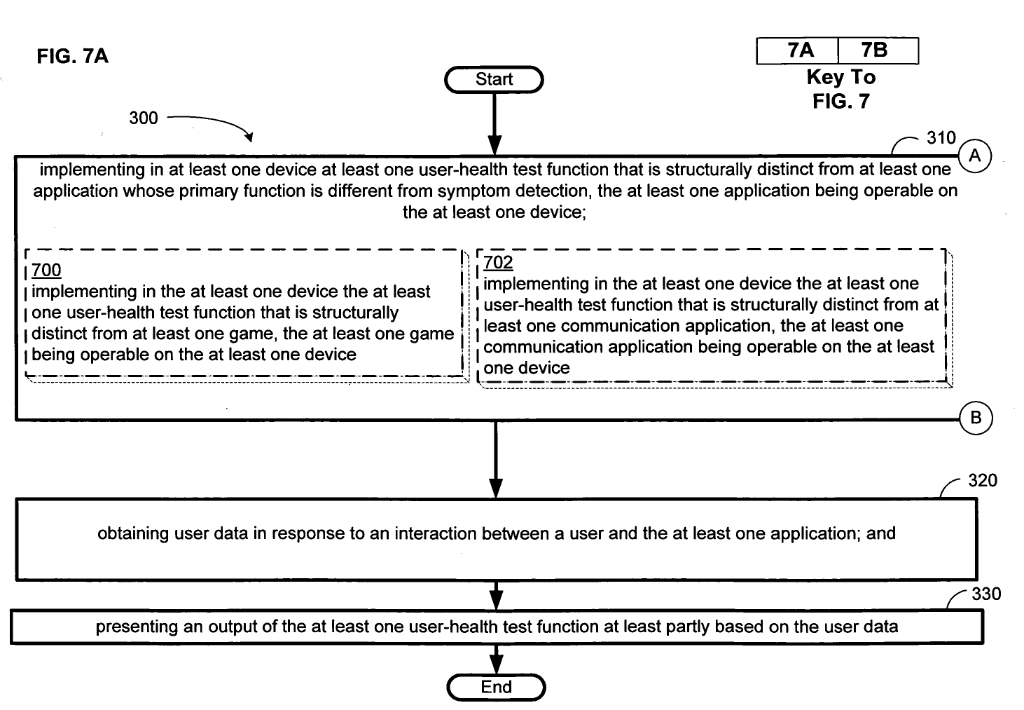

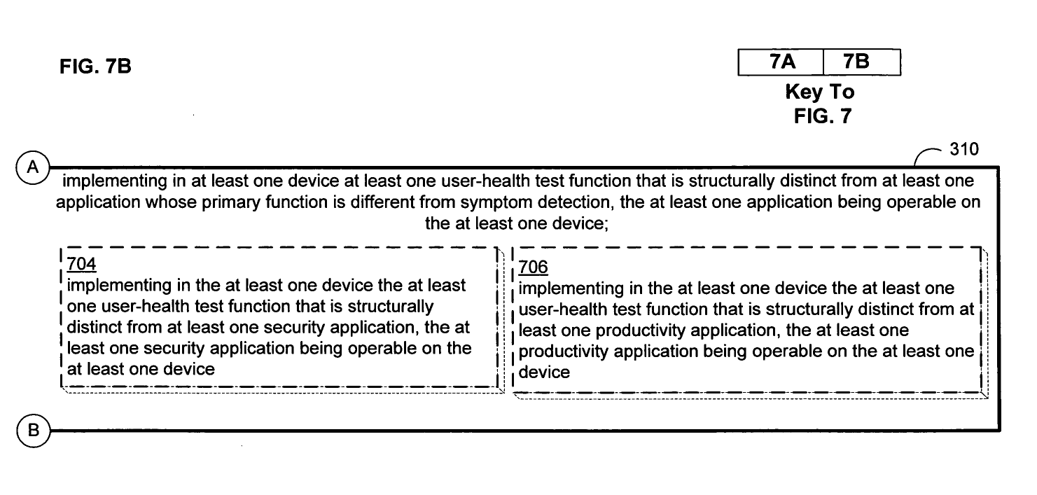

FIG. 7 illustrates alternative embodiments of the example operational flow 300 of FIG. 3. FIG. 7 illustrates example embodiments where the implementing operation 310 may include at least one additional operation. Additional operations may include operation 700, 702, 704, and/or operation 706.

Operation 700 depicts implementing in the at least one device the at least one user-health test function that is structurally distinct from at least one game, the at least one game being operable on the at least one device. For example, a device 102 may have installed on it a user-health test unit 104 that is structurally distinct from at least one game 144. The user-health test unit 104 can receive user data 116 from an interaction between user 110 and the game 144, whose primary function is different from symptom detection, the game 144 being operable on the at least one device 102. Such a game 144 may generate user data 116 via a user input device 146 or a user monitoring device 148. Examples of a user input device 146 include a text entry device such as a keyboard, a pointing device such as a mouse, a touchscreen, or the like. Examples of a user monitoring device 148 include a microphone, a photography device, a video device, or the like.

Examples of a game 144 may include a computer game such as, for example, solitaire, puzzle games, role-playing games, first-person shooting games, strategy games, sports games, racing games, adventure games, or the like. Such games may be played offline or through a network (e.g., online games).

Operation 702 depicts implementing in the at least one device the at least one user-health test function that is structurally distinct from at least one communication application, the at least one communication application being operable on the at least one device. For example, a user-health test unit 104 may be implemented over a network 202 on the at least one device 102, the user-health test unit being structurally distinct from at least one communication application 150 operable on the at least one device 102. The user-health test unit 104 can receive user data 116 from an interaction between user 110 and the communication application 150, whose primary function is different from symptom detection. Such a communication application 150 may generate user data 116 via a user input device 146 or a user monitoring device 148.

Examples of a communication application 150 may include various forms of one-way or two-way information transfer, typically to, from, between, or among devices. Some examples of communications applications include: an email program, a telephony application, a videocommunications function, an internet or other network messaging program, a cell phone communication application, or the like. Such a communication application may operate via text, voice, video, or other means of communication, combinations of these, or other means of communication.

Operation 704 depicts implementing in the at least one device the at least one user-health test function that is structurally distinct from at least one security application, the at least one security application being operable on the at least one device. For example, a user-health test unit 104 may be implemented over a network 202 on a device 102, the user-health test unit being structurally distinct from at least one security application 152 operable on the at least one device 102. The user-health test unit 104 can receive user data 116 from an interaction between user 110 and the security application 152, whose primary function is different from symptom detection. Such a security application 152 may generate user data 116 via a user input device 146 or a user monitoring device 148.

Examples of a security application 152 may include a password entry program, a code entry system, a biometric identification application, a video monitoring system, or the like.

Operation 706 depicts implementing in the at least one device the at least one user-health test function that is structurally distinct from at least one productivity application, the at least one productivity application being operable on the at least one device. For example, a user-health test unit 104 may be implemented over a network 202 on the at least one device 102, the user-health test unit being structurally distinct from at least one productivity application 154 operable on the at least one device 102. The user-health test unit 104 can receive user data 116 from an interaction between user 110 and the productivity application 154, whose primary function is different from symptom detection. Such a productivity application 154 may generate user data 116 via a user input device 146 or a user monitoring device 148.

Examples of a productivity application 154 may include a word processing program, a spreadsheet program, business software, or the like.

FIG. 8 illustrates alternative embodiments of the example operational flow 300 of FIG. 3. FIG. 8 illustrates example embodiments where the obtaining operation 320 may include at least one additional operation. Additional operations may include operation 800, 802, 804, 806, 808, 810, 812, and/or operation 814.

Operation 800 depicts obtaining user reaction time data in response to the interaction between the user and the at least one application. For example, a device 102 may have installed on it at least one application 106 that is structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user reaction time data, for example, in response to an interaction between the user and the at least one application 106, for example via user interface 108.

Examples of reaction time data may include speed of a user 110's response to a prompting icon on a display, for example by clicking with a mouse or other pointing device or by some other response mode. For example, within a game situation a user may be prompted to click on a target as a test of alertness or awareness. Data may be collected once or many times for this task. A multiplicity of data points indicating a change in reaction time may be indicative of a change in alertness, awareness, neglect, construction, memory, hearing, or other user-health attribute as discussed above.

Operation 802 depicts obtaining user movement data in response to the interaction between the user and the at least one application. For example, a device 102 may have installed on it at least one application 106 that is structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user movement data in response to an interaction between the user and the at least one application 106, for example via user interface 108.

An example of user movement data may include data from a pointing device when a user is prompted to activate or click a specific area on a display to test, for example, visual field range or motor skill function. Another example is visual data of a user's body, for example during a videoconference, wherein changes in facial movement, limb movement, or other body movements are detectable, as discussed above.

Operation 804 depicts obtaining user cognitive function data in response to the interaction between the user and the at least one application. For example, a device 102 may have installed on it at least one application 106 that is structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user cognitive function data in response to an interaction between the user and the at least one application 106, for example via user interface 108.

An example of user cognitive function data may include data from a text or number input device or user monitoring device when a user is prompted to, for example, spell, write, speak, or calculate in order to test, for example, alertness, ability to calculate, speech, motor skill function, or the like, as discussed above.

Operation 806 depicts obtaining user memory function data in response to the interaction between the user and the at least one application. For example, a device 102 may have installed on it at least one application 106 that is structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user memory function data in response to an interaction between the user and the at least one application 106, for example via user interface 108.

An example of user memory function data may include data from a text or number input device or user monitoring device when a user is prompted to, for example, spell, write, speak, or calculate in order to test, for example, short-term memory, long-term memory, or the like, as discussed above.

Operation 808 depicts obtaining user voice or speech data in response to the interaction between the user and the at least one application. For example, a device 102 may have installed on it at least one application 106 that is structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user voice or speech data in response to an interaction between the user and the at least one application 106, for example via user interface 108.

An example of user voice or speech data may include data from a speech or voice input device or user monitoring device, such as a telephonic device or a video communication device with sound receiving/transmission capability, for example when a user task requires, for example, speaking, singing, or other vocalization, as discussed above.

Operation 810 depicts obtaining user eye movement data in response to the interaction between the user and the at least one application. For example, a device 102 may have installed on it at least one application 106 that is-structurally distinct from a user-health test unit 104 that is installed on an external device 160 that has access to the user data 116 generated by device 102 on which the application 106 is installed. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104, either resident on device 102 or in this example resident on an external device 160 that communicates with device 102, can obtain user eye movement data in response to an interaction between the user and the at least one application 106, for example via user interface 108.

An example of user eye movement data may include data from a user monitoring device, such as a video communication device, for example, when a user task requires tracking objects on a display, reading, or during resting states between activities in an application, as discussed above. A further example includes pupillary reflex data from the user at rest or during an activity required by an application or user-health test function.

Operation 812 depicts obtaining user internet usage data in response to the interaction between the user and the at least one application. For example, a device 102 may have installed on it at least one application 106 that is structurally distinct from a user-health test unit 104 that is also installed on the device 102. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104 can obtain user internet usage data in response to an interaction between the user 110 and the at least one application 106, for example via user interface 108.

An example of user internet usage data may include data from a user's pointing device (including ability to click on elements of a web page, for example), browser history/function (including sites visited, ability to navigate from one site to another, ability to go back to a previous website if prompted, or the like), monitoring device, such as a video communication device, for example, when an application task or user-health test function task requires interaction with a web browser. Such data may indicate cognitive, memory, or motor skill function impairment, or the like, as discussed above. Other examples of internet usage data may include data from a user's offline interaction with internet content obtained while online.

Operation 814 depicts obtaining user image data in response to the interaction between the user and the at least one application. For example, a device 102 may have installed on it at least one application 106 that is structurally distinct from a user-health test unit 104 that is also installed on the device 102. The user-health test unit 104 can receive user data 116 from an interaction between user 110 and the application 106, whose primary function is different from symptom detection, the application 106 being operable on the device 102. Such an application 106 may generate user data 116 via a user input device 146 or a user monitoring device 148. User-health test unit 104 can obtain user image data in response to an interaction between the user 110 and the at least one application 106, for example via user interface 108.

An example of user image data may include data from a user's video capture device, monitoring device, such as a video communication device, for example, when a user inputs a photograph or video when using an application, or when a user's image is captured when communicating via a photography or video-based application. Other examples of user image data may include biometric data such as facial pattern data, eye scanning data, or the like. Such user image data may indicate, for example, alertness, attention, motor skill function impairment, or the like, as discussed above.

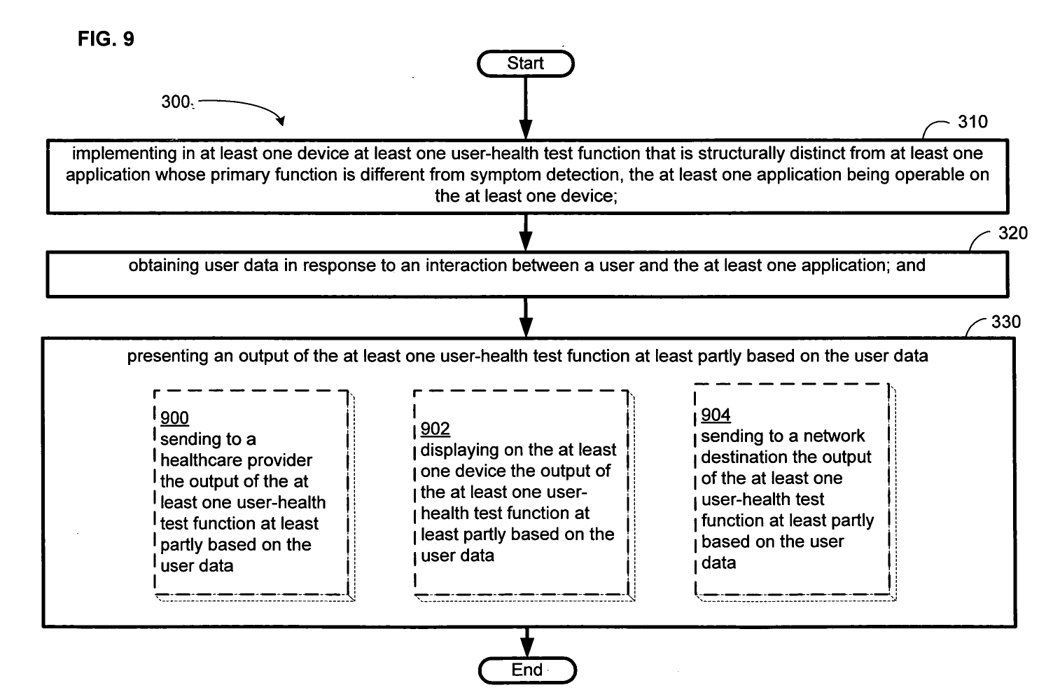

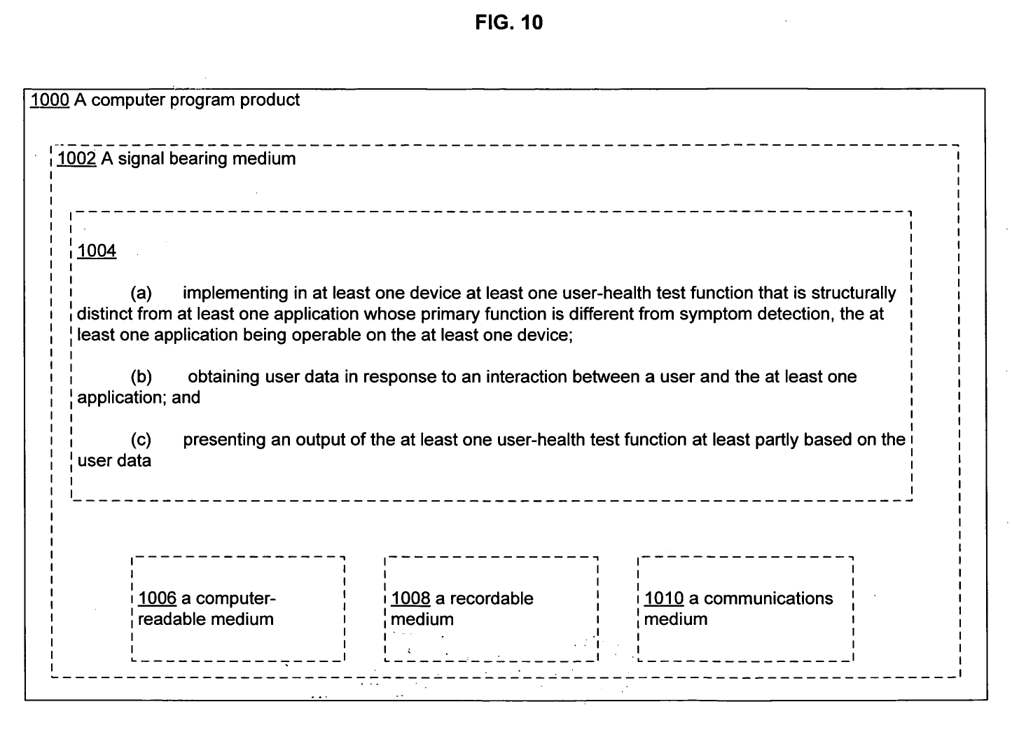

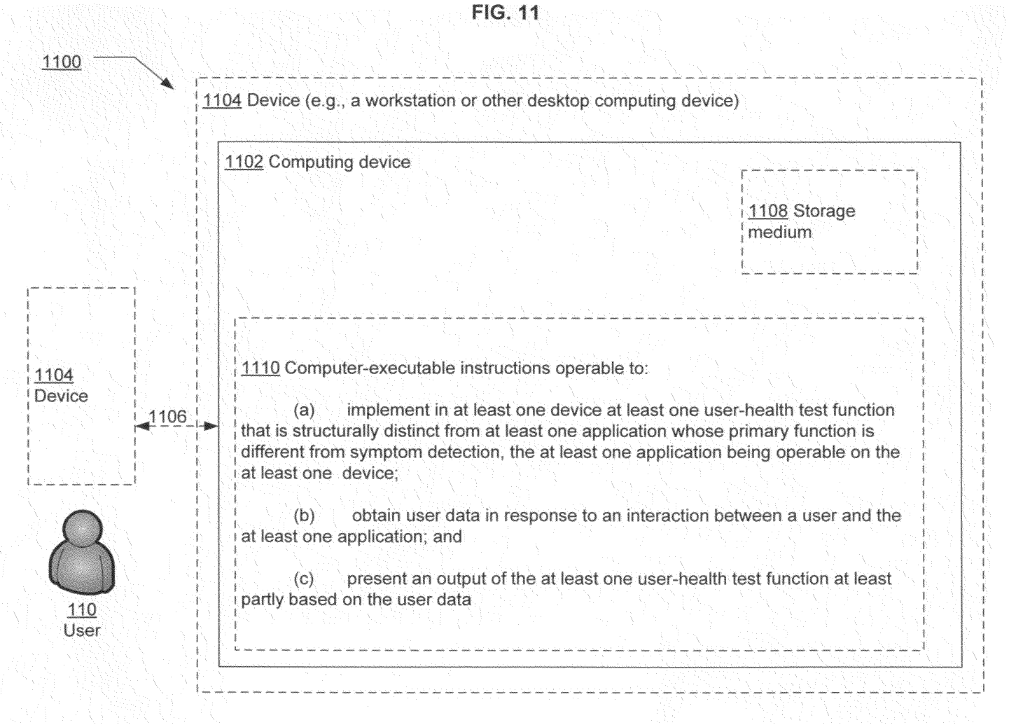

FIG. 9 illustrates alternative embodiments of the example operational flow 300 of FIG. 3. FIG. 9 illustrates example embodiments where the presenting operation 330 may include at least one additional operation. Additional operations may include operation 900, 902, and/or operation 904.

Operation 900 depicts sending to a healthcare provider the output of the at least one user-health test function at least partly based on the user data. For example, a user-health test unit 104 may be installed on an external device 160 with access to user data 116 generated by a user 10 interaction with application 106 whose primary function is different from symptom detection, application 106 being operable on device 102. The user-health test unit 104 can receive user data 116, and based at least partly on user data 116 can send an output to a healthcare provider 210, for example, over a network 202. Examples of a healthcare provider 210 may include physicians, therapists, nurses, counselors, hospitals, health maintenance organizations, or the like.

Operation 902 depicts displaying on the at least one device the output of the at least one user-health test function at least partly based on the user data. For example, a user-health test unit 104 may be installed on an external device 160 with access to user data 116 generated by a user 110 interaction with application 106 whose primary function is different from symptom detection, application 106 being operable on device 102. The user-health test unit 104 can receive user data 116, and based at least partly on user data 116 can send an output to a display on the device 102. Examples of outputs displayed on a device 102 may include an alert message in an email application, a notification window in a desktop area of an operating system running on the device, a program window that shows the output to the user in the context of the user-health test function, or the like.

Examples of an output of a user-health test function or user-health test unit may include baseline user attributes such as reaction time, motor skill function, visual field range, or the like. Further examples of an output of a user-health test function or user-health test unit 104 may include an aggregation or distillation of user data acquired over a period of time. Statistical filters may be applied to user data by the user-health test function, or profiles corresponding to various health-related problems may be matched with user data or a distillation of user data.

Operation 904 depicts sending to a network destination the output of the at least one user-health test function at least partly based on the user data. For example, a user-health test unit 104 may be installed on an external device 160 with access to user data 116 generated by a user 110 interaction with application 106 whose primary function is different from symptom detection, application 106 being operable on device 102. The user-health test unit 104 can receive user data 116, and based at least partly on user data 116 can send an output to a network destination 206, for example, over a network 202. Examples of network destinations may include devices including computers connected to, for example, a home network accessible by parents of a child user, devices connected by the internet to the device 102 or user interface 108, or the like.

FIG. 10 illustrates a partial view of an example computer program product 1000 that includes a computer program 1004 for executing a computer process on a computing device. An embodiment of the example computer program product 1000 is provided using a signal bearing medium 1002, and may include one or more instructions for implementing in at least one device at least one user-health test function that is structurally distinct from at least one application whose primary function is different from symptom detection, the at least one application being operable on the at least one device; one or more instructions for obtaining user data in response to an interaction between a user and the at least one application; and one or more instructions for presenting an output of the at least one user-health test function at least partly based on the user data. The one or more instructions may be, for example, computer executable and/or logic-implemented instructions. In one implementation, the signal-bearing medium 1002 may include a computer-readable medium 1006. In one implementation, the signal bearing medium 1002 may include a recordable medium 1008. In one implementation, the signal bearing medium 1002 may include a communications medium 1010.

FIG. 11 illustrates an example system 1100 in which embodiments may be implemented. The system 1100 includes a computing system environment. The system 1100 also illustrates the user 110 using a device 1104, which is optionally shown as being in communication with a computing device 1102 by way of an optional coupling 1106. The optional coupling 1106 may represent a local, wide-area, or peer-to-peer network, or may represent a bus that is internal to a computing device (e.g., in example embodiments in which the computing device 1102 is contained in whole or in part within the device 1104). A storage medium 1108 may be any computer storage media.

The computing device 1102 includes computer-executable instructions 1110 that when executed on the computing device 1102 cause the computing device 1102 to (a) implement in at least one device at least one user-health test function that is structurally distinct from at least one application whose primary function is different from symptom detection, the at least one application being operable on the at least one device; (b) obtain user data in response to an interaction between the user and the at least one application; and (c) present an output of the user-health test function at least partly based on the user data. As referenced above and as shown in FIG. 11, in some examples, the computing device 1102 may optionally be contained in whole or in part within the device 1104.

In FIG. 11, then, the system 1100 includes at least one computing device (e.g., 1102 and/or 1104). The computer-executable instructions 1110 may be executed on one or more of the at least one computing device. For example, the computing device 1102 may implement the computer-executable instructions 1110 and output a result to (and/or receive data from) the computing device 1104. Since the computing device 1102 may be wholly or partially contained within the computing device 1104, the device 1104 also may be said to execute some or all of the computer-executable instructions 1110, in order to be caused to perform or implement, for example, various ones of the techniques described herein, or other techniques.

The device 1104 may include, for example, a portable computing device, workstation, or desktop computing device. In another example embodiment, the computing device 1102 is operable to communicate with the device 1104 associated with the user 110 to receive information about the input from the user 110 for performing data access and data processing and presenting an output of the user-health test function at least partly based on the user data.

Although a user 110 is shown/described herein as a single illustrated figure, those skilled in the art will appreciate that a user 110 may be representative of a human user, a robotic user (e.g., computational entity), and/or substantially any combination thereof (e.g., a user may be assisted by one or more robotic agents). In addition, a user 110, as set forth herein, although shown as a single entity may in fact be composed of two or more entities. Those skilled in the art will appreciate that, in general, the same may be said of “sender” and/or other entity-oriented terms as such terms are used herein.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

All of the above U.S. patents, U.S. patent application publications, U.S. patent applications, foreign patents, foreign patent applications and non-patent publications referred to in this specification and/or listed in any Application Data Sheet are incorporated herein by reference, to the extent not inconsistent herewith.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

1. A method comprising:
implementing in at least one device at least one user-health test function that is structurally distinct from at least one application whose primary function is different from symptom detection, the at least one application being operable on the at least one device;
obtaining user data in response to an interaction between a user and the at least one application; and
presenting an output of the at least one user-health test function at least partly based on the user data.

2. (canceled)

3. The method of claim 1 wherein the implementing in at least one device at least one user-health test function that is structurally distinct from at least one application whose primary function is different from symptom detection, the at least one application being operable on the at least one device comprises:
implementing in the at least one device at least one alertness or attention test function that is structurally distinct from the at least one application whose primary function is different from symptom detection, the at least one application being operable on the at least one device.

4. The method of claim 1 wherein the implementing in at least one device at least one user-health test function that is structurally distinct from at least one application whose primary function is different from symptom detection, the at least one application being operable on the at least one device comprises:
implementing in the at least one device at least one memory test function that is structurally distinct from the at least one application whose primary function is different from symptom detection, the at least one application being operable on the at least one device.

5. The method of claim 1 wherein the implementing in at least one device at least one user-health test function that is structurally distinct from at least one application whose primary function is different from symptom detection, the at least one application being operable on the at least one device comprises:
implementing in the at least one device at least one speech test function that is structurally distinct from the at least one application whose primary function is different from symptom detection, the at least one application being operable on the at least one device.

6. The method of claim 1 wherein the implementing in at least one device at least one user-health test function that is structurally distinct from at least one application whose primary function is different from symptom detection, the at least one application being operable on the at least one device comprises:
implementing in the at least one device at least one calculation test function that is structurally distinct from the at least one application whose primary function is different from symptom detection, the at least one application being operable on the at least one device.

7. The method of claim 1 wherein the implementing in at least one device at least one user-health test function that is structurally distinct from at least one application whose primary function is different from symptom detection, the at least one application being operable on the at least one device comprises:
implementing in the at least one device at least one neglect or construction test function that is structurally distinct from the at least one application whose primary function is different from symptom detection, the at least one application being operable on the at least one device.

8. The method of claim 1 wherein the implementing in at least one device at least one user-health test function that is structurally distinct from at least one application whose primary function is different from symptom detection, the at least one application being operable on the at least one device comprises:
implementing in the at least one device at least one task sequencing test function that is structurally distinct from the at least one application whose primary function is different from symptom detection, the at least one application being operable on the at least one device.

9. The method of claim 1 wherein the implementing in at least one device at least one user-health test function that is structurally distinct from at least one application whose primary function is different from symptom detection, the at least one application being operable on the at least one device comprises:
implementing in the at least one device at least one visual field test function that is structurally distinct from the at least one application whose primary function is different from symptom detection, the at least one application being operable on the at least one device.

10. The method of claim 1 wherein the implementing in at least one device at least one user-health test function that is structurally distinct from at least one application whose primary function is different from symptom detection, the at least one application being operable on the at least one device comprises:
implementing in the at least one device at least one pupillary reflex or eye movement test function that is structurally distinct from the at least one application whose primary function is different from symptom detection, the at least one application being operable on the at least one device.

11. The method of claim 1 wherein the implementing in at least one device at least one user-health test function that is structurally distinct from at least one application whose primary function is different from symptom detection, the at least one application being operable on the at least one device comprises:
implementing in the at least one device at least one face pattern test function that is structurally distinct from the at least one application whose primary function is different from symptom detection, the at least one application being operable on the at least one device.

12. The method of claim 1 wherein the implementing in at least one device at least one user-health test function that is structurally distinct from at least one application whose primary function is different from symptom detection, the at least one application being operable on the at least one device comprises:
implementing in the at least one device at least one hearing test function that is structurally distinct from the at least one application whose primary function is different from symptom detection, the at least one application being operable on the at least one device.

13. The method of claim 1 wherein the implementing in at least one device at least one user-health test function that is structurally distinct from at least one application whose primary function is different from symptom detection, the at least one application being operable on the at least one device comprises:
implementing in the at least one device at least one voice test function that is structurally distinct from the at least one application whose primary function is different from symptom detection, the at least one application being operable on the at least one device.

14. The method of claim 1 wherein the implementing in at least one device at least one user-health test function that is structurally distinct from at least one application whose primary function is different from symptom detection, the at least one application being operable on the at least one device comprises:
implementing in the at least one device at least one motor skill test function that is structurally distinct from the at least one application whose primary function is different from symptom detection, the at least one application being operable on the at least one device.

15. The method of claim 1 wherein the implementing in at least one device at least one user-health test function that is structurally distinct from at least one application whose primary function is different from symptom detection, the at least one application being operable on the at least one device comprises:
implementing in the at least one device at least one body movement test function that is structurally distinct from the at least one application whose primary function is different from symptom detection, the at least one application being operable on the at least one device.

16. The method of claim 1 wherein the implementing in at least one device at least one user-health test function that is structurally distinct from at least one application whose primary function is different from symptom detection, the at least one application being operable on the at least one device comprises:
implementing in the at least one device the at least one user-health test function that is structurally distinct from at least one game, the at least one game being operable on the at least one device.

17. The method of claim 1 wherein the implementing in at least one device at least one user-health test function that is structurally distinct from at least one application whose primary function is different from symptom detection, the at least one application being operable on the at least one device comprises:
implementing in the at least one device the at least one user-health test function that is structurally distinct from at least one communication application, the at least one communication application being operable on the at least one device.

18. The method of claim 1 wherein the implementing in at least one device at least one user-health test function that is structurally distinct from at least one application whose primary function is different from symptom detection, the at least one application being operable on the at least one device comprises:
implementing in the at least one device the at least one user-health test function that is structurally distinct from at least one security application, the at least one security application being operable on the at least one device.

19. The method of claim 1 wherein the implementing in at least one device at least one user-health test function that is structurally distinct from at least one application whose primary function is different from symptom detection, the at least one application being operable on the at least one device comprises:
implementing in the at least one device the at least one user-health test function that is structurally distinct from at least one productivity application, the at least one productivity application being operable on the at least one device.

20. The method of claim 1 wherein the obtaining user data in response to an interaction between a user and the at least one application comprises:
obtaining user reaction time data in response to the interaction between the user and the at least one application.

21. The method of claim 1 wherein the obtaining user data in response to an interaction between a user and the at least one application comprises:
obtaining user movement data in response to the interaction between the user and the at least one application.

22. The method of claim 1 wherein the obtaining user data in response to an interaction between a user and the at least one application comprises:
obtaining user cognitive function data in response to the interaction between the user and the at least one application.

23. The method of claim 1 wherein the obtaining user data in response to an interaction between a user and the at least one application comprises:
obtaining user memory function data in response to the interaction between the user and the at least one application.

24. The method of claim 1 wherein the obtaining user data in response to an interaction between a user and the at least one application comprises:
obtaining user voice or speech data in response to the interaction between the user and the at least one application.

25. The method of claim 1 wherein the obtaining user data in response to an interaction between a user and the at least one application comprises:
obtaining user eye movement data in response to the interaction between the user and the at least one application.

26. The method of claim 1 wherein the obtaining user data in response to an interaction between a user and the at least one application comprises:
obtaining user internet usage data in response to the interaction between the user and the at least one application.

27. The method of claim 1 wherein the obtaining user data in response to an interaction between a user and the at least one application comprises:
obtaining user image data in response to the interaction between the user and the at least one application.

28-30. (canceled)

31. A system comprising:
means for implementing in at least one device at least one user-health test function that is structurally distinct from at least one application whose primary function is different from symptom detection, the at least one application being operable on the at least one device;
means for obtaining user data in response to an interaction between a user and the at least one application; and
means for presenting an output of the at least one user-health test function at least partly based on the user data.

32. The system of claim 31 wherein the means for implementing in at least one device at least one user-health test function that is structurally distinct from at least one application whose primary function is different from symptom detection, the at least one application being operable on the at least one device comprises:
means for implementing in at least one desktop computing device the at least one user-health test function that is structurally distinct from the at least one application whose primary function is different from symptom detection, the at least one application being operable on the at least one device.

33-57. (canceled)

58. The system of claim 31 wherein the means for presenting an output of the at least one user-health test function at least partly based on the user data comprises:
means for sending to a healthcare provider the output of the at least one user-health test function at least partly based on the user data.

59. The system of claim 31 wherein the means for presenting an output of the at least one user-health test function at least partly based on the user data comprises:
means for displaying on the at least one device the output of the at least one user-health test function at least partly based on the user data.

60. The system of claim 31 wherein the means for presenting an output of the at least one user-health test function at least partly based on the user data comprises:
means for sending to a network destination the output of the at least one user-health test function at least partly based on the user data.

61. A computer program product comprising:
a signal-bearing medium bearing
(a) one or more instructions for implementing in at least one device at least one user-health test function that is structurally distinct from at least one application whose primary function is different from symptom detection, the at least one application being operable on the at least one device;
(b) one or more instructions for obtaining user data in response to an interaction between a user and the at least one application; and
(c) one or more instructions for presenting an output of the at least one user-health test function at least partly based on the user data.

62. The computer program product of claim 61, wherein the signal-bearing medium includes a computer-readable medium.

63. The computer program product of claim 61, wherein the signal-bearing medium includes a recordable medium.

64. The computer program product of claim 61, wherein the signal-bearing medium includes a communications medium.

65. A system comprising:
a computing device; and
instructions that when executed on the computing device cause the computing device to
(a) implement in at least one device at least one user-health test function that is structurally distinct from at least one application whose primary function is different from symptom detection, the at least one application being operable on the at least one device;
(b) obtain user data in response to an interaction between the user and the at least one application; and
(c) present an output of the user-health test function at least partly based on the user data.

66. The system of claim 65 wherein the computing device comprises:
one or more of a personal digital assistant (PDA), a personal entertainment device, a mobile phone, a laptop computer, a tablet personal computer, a networked computer, a computing system comprised of a cluster of processors, a computing system comprised of a cluster of servers, a workstation computer, and/or a desktop computer.

67. The system of claim 65 wherein the computing device is operable to obtain user data in response to the interaction between the user and the at least one application and present the output of the at least one user-health test function at least partly based on the user data from at least one memory.
